(19) United States
(12) Patent Application Publication
(10) Pub. No.: US 2022/0071494 A1
(43) Pub. Date: Mar. 10, 2022

(54) TEMPERATURE MEASUREMENT SYSTEMS AND METHODS USING MAGNETIC RESONANCE IMAGING

(71) Applicants: Zbigniew Celinski, Colorado Springs, CO (US); Janusz Hankiewicz, Colorado Springs, CO (US); Robert Camley, Colorado Springs, CO (US)

(72) Inventors: Zbigniew Celinski, Colorado Springs, CO (US); Janusz Hankiewicz, Colorado Springs, CO (US); Robert Camley, Colorado Springs, CO (US)

(21) Appl. No.: 17/469,726

(22) Filed: Sep. 8, 2021

Related U.S. Application Data
(60) Provisional application No. 63/093,989, filed on Oct. 20, 2020.
(60) Provisional application No. 63/075,669, filed on Sep. 8, 2020.

(51) Int. Cl. A61B 5/01; A61B 18/02; A61B 5/055; A61K 41/00; A61K 9/00; A61P 35/00; G01R 33/50

(52) U.S. Cl. CPC A61B 5/015; A61B 18/0218; A61B 5/055; G01R 33/50; A61K 9/0024; A61P 35/00; A61K 41/0052

(57) ABSTRACT

Provided are a system and a method for determining the temperature of a body by imaging a hydrogen proton-rich material positioned within the body using nuclear magnetic resonance imaging. A method to increase changes in the MRI signal strength as a function of temperature, thus improving temperature sensitivity, is also provided. The system and method employ polymers having mechanical stability and magnetic image brightness at low temperatures of between 0° C. and −65° C. or high temperatures of between +37° C. and +80° C.

### CROSS-REFERENCE TO RELATED APPLICATIONS

This application claims the benefit of U.S. Provisional Patent Application Ser. No. 63/093,989 filed on Oct. 20, 2020, and U.S. Provisional Patent Application Ser. No. 63/075,669 filed on Sep. 8, 2020, the disclosures of which are incorporated herein by reference.

### GOVERNMENT SUPPORT

This invention was made with Government support under Grant No. SBIR-1843616 awarded by the National Science Foundation. The Government has certain rights in the invention.

### FIELD OF THE INVENTION

The present invention relates generally to magnetic resonance (MR) imaging and MR techniques for temperature measurement. The present invention relates more specifically to MR thermometry for use in cryoablation and high temperature ablation of diseased tissue.

### BACKGROUND OF THE INVENTION

Magnetic Resonance Imaging (MRI) is used to guide a variety of interventional surgeries, including surgical procedures for treating cancers, resulting in less invasive procedures and significantly reduced side effects. Early techniques often killed tumors by heating them above +45° C. (heat or thermal ablation), with heating provided, for example, by a laser beam guided into the tumor by a glass fiber and positioned by MRI.

Recently there has been a move to killing tumors by freezing instead of by heating the cancerous cells. MRI-guided cryoablation is an interventional procedure which kills tumors by freezing the cells in the tumor. Cryoablation surgery utilizes a probe to locally freeze the tumor, creating an ice ball, and resulting in direct damage to tumor cells by a repeated process of freezing and thawing. Cells usually die at temperatures between −20° C. and −50° C. due to membrane ruptures, cellular dehydration and local ischemia. The positioning of the needle applicator is guided by MRI.

Cryoablation surgery guided by magnetic resonance imaging (MRI) is a minimally invasive technology used to treat a wide variety of cancers, significantly reducing side effects, complications and recovery time compared to current treatments. During the cryoablation procedure, near real-time temperature information is necessary to monitor changes in tissue temperatures to ensure killing the tumor while leaving the adjacent tissue undamaged. The current method for obtaining temperature maps in MRI, proton resonance frequency shifts (PRF) fails completely below the freezing point of water.

MRI guided cryoablation provides multiple advantages such as reduced side effects, identification of the edges of the tumor, and localization of the ice ball. Unfortunately, at temperatures below 0° C., standard MRI fails to provide any actual image of the frozen tissue due to a significant linewidth broadening. Basically, the MR image of the ice ball simply turns black. In other words, the surgeon can visualize an ice ball, but cannot ascertain the temperature inside the ice ball such as the core temperature of the tissue to be ablated. In reality, there is a temperature gradient inside the ice ball reflecting a different temperature at the applicator tip relative to the periphery of the ice ball. For example, the peripheral cortex of the ice ball may be at or near 0° C., but the inner medullary portions of the ball are expected to be at colder temperatures. Since one must reach temperatures well below freezing to ensure the death of the tumor cells or other diseased tissue, this black ice ball under MRI presents a significant problem of a critical lack of temperature information required for cryoablation.

Proton resonance frequency (PRF) shift, developed by De Poorter et al. (Magn. Reson. Med., 33: 74-81 (1995)) is a commonly used method used to measure tissue temperature in thermal ablations. However, PRF is completely ineffective at low temperatures, as evidenced by Rieke, V., and Pauly, K. B., J. Magn. Reson. Imaging, 27: 376-390 (2008); Odéen, H., and Parker, D. L., Prog. Nucl. Reson. Spectrosc., 110: 34-61 (2019), which are incorporated herein by reference. The ineffectiveness of PRF for temperature measurement during cryoablation is due to a large increase in linewidth for hydrogen protons as the material freezes.

Ultrashort echo-time MRI sequencing is another method that does allow visualization of temperature inside the ice ball, but only at temperatures above −40° C., which is insufficiently cold for effective cryoablation. Furthermore, ultrashort echo-time MRI sequencing requires more than 1 minute of acquisition time, as evidenced by Overduin et al., J. Magn. Reson. Imaging, 44: 1572-1579 (2016), which is incorporated herein by reference. Thus, ultrashort echo-time MRI sequencing is also not useful in clinical settings to measure very low temperatures necessary for tissue cryoablation.

The lack of real time knowledge about the temperature within the to-be-ablated tissue produces multiple unwanted outcomes, including temperatures within the ice ball that are not low enough to completely kill the tumor tissue, resulting in a recurrence of the cancer, and the generation of ice balls that extend too far beyond the tumor and unnecessarily damage healthy tissue. Thus, there exists a significant need for MR systems and methods to accurately measure very low temperatures within disease tissue in real time to enhance the safety and effectiveness of cryoablation therapy.

### SUMMARY

Disclosed are a system and a method for determining the temperature of a cold object using magnetic resonance imaging. In one embodiment, the cold object has a temperature that is ≤−30° C., preferably ≤−40° C. In one embodiment, the cold object is a target tissue, such as e.g., a tumor or other neoplastic tissue during cryoablation therapy. The subject system and method can be used during MRI-guided cryoablation operations, significantly reducing both procedure time and cost, and making cryoablation surgery safer and more effective.

Using both MRI and nuclear magnetic resonance (NMR), the inventor has made the surprising discovery that certain polymers show nuclear relaxation times with a significant and monotonic dependence on temperature. These polymers were observed to exhibit a temperature-dependent brightness in MRI in the temperature range of about −60° C. to +60° C., with a thermal resolution of better than 5° C. across 3 mm and an acquisition time of less than 5 seconds.

In a first aspect, the invention provides a method for determining the temperature of a target body by (a) placing a temperature-stable hydrogen proton-rich material, such as, e.g., a polymer, into or proximate to a target body, (b) cooling the target body to a temperature below the freezing point of water in the target body, (c) and imaging the a temperature-stable hydrogen proton-rich material using MM, such that the level of brightness of the temperature-stable hydrogen proton-rich material within the target body correlates with the temperature of the target body.

In one embodiment, the target body is a tissue within a patient. In a preferred embodiment, the tissue is a disease tissue including inter alia tumor, dysplasia, neoplasia, hyperplasia, carcinoma, sarcoma, and conducting tissue such as e.g., conductance cells at or near the atrioventricular node.

In one embodiment, the temperature below the freezing point of water in the target body is ≤0° C., ≤−10° C., ≤−20° C., ≤−30° C., ≤−40° C., ≤−50° C., ≤−60° C., ≤−70° C., or ≤−75° C. In one embodiment, temperature below the freezing point of water in the target body is a temperature that kills target cells after one or more freeze-thaw cycles.

In one embodiment, the temperature-stable hydrogen proton-rich material is a polymer, more preferably a biocompatible polymer. In one embodiment, the temperature-stable hydrogen proton-rich material is mechanically stable from room temperature to ≤−65° C. or ≤−75° C. In one embodiment, the temperature-stable hydrogen proton-rich material has no freezing transition in the temperature range of from about −75° C. to about +65° C., or about −50° C. to about +65° C. In one embodiment, the temperature-stable hydrogen proton-rich material contains a high abundance of hydrogen protons to enable the magnetic resonance image to be detectable and brighter than the surrounding target. In one embodiment, the temperature-stable hydrogen proton-rich material has strong and regular/monotonic temperature dependence of the nuclear relaxation times T₁, T₂, or T₂*, and the nuclear relaxation times T₁, T₂, or T₂* are in the range of about 5 ms to about 1,500 ms over the entire temperature range.

In a more specific embodiment, the temperature-stable hydrogen proton-rich material (a) is mechanically stable from room temperature to ≤−65° C. or ≤−75° C.; (b) has no freezing transition in the temperature range of from about −75° C. to about +50° C., or about −50° C. to about +50° C.; (c) contains a high abundance of hydrogen protons to enable the magnetic resonance image to be detectable and brighter than the surrounding target; (d) has strong and regular/monotonic temperature dependence of the nuclear relaxation times T₁, or T₂, or T₂*; and (e) has nuclear relaxation times T₁, T₂, or T₂* in the range of about 5 ms to about 1,500 ms over the entire temperature range.

In an alternative embodiment, the temperature-stable hydrogen proton-rich material contains a base material and a magnetic material. Here, the base material is a polymer with a narrow NMR linewidth and weak temperature dependence in the range of about 0° C. to about −65° C. The magnetic particles confer to the temperature-stable hydrogen proton-rich material a linewidth having a strong temperature dependence. In one embodiment, the undoped base material has a weak temperature dependence of the nuclear relaxation times T₁ and T₂, and the base material once doped with particles has a shortened T₂* and increased thermal dependence.

In one embodiment, the magnetic particles change in magnetization with change in temperature over a temperature range of at least from about +37° C. to about −50° C. (or about −75° C. or about −65° C.) in the field of the MM scanner. In one embodiment, the magnetic material contains or consists of magnetic materials with a net magnetization, such as iron oxide-containing materials, such as, e.g., Cu_{0.24}Zn_{0.76}Fe₂O₄ with a diameter of ranging from 5 nm to 3 microns. In another embodiment, the magnetic material contains or consists of Mn_{0.48}Zn_{0.46}Fe_{2.06}O₄ with a diameter of ranging from 5 nm to 3 microns. In one embodiment, the concentration of magnetic particles in the base material is about 0.05 mM to about 3 mM.

In still other embodiments, the magnetic particle is any magnetic particle now known or yet to be discovered. In still other embodiments, useful magnetic particles include paramagnetic materials, such as, e.g., gadolinium (a known MRI contrast medium), metalloporphrins (e.g., manganese(III) tetra-[4-sulfanatophenyl] porphyrin), and the like. Magnetic particles useful in the practice of this invention are generally disclosed in the following references: (i) U.S. Patent Application Publication No. US 2018/0117186 A1, (ii) “Structural, Magnetic and Toxicity Studies of Ferrite Particles Employed as Contrast Agents for Magnetic Resonance Imaging Thermometry”, Journal of Magnetism and Magnetic Materials, 497, (2020), 165981, (iii) “Nano-Sized Ferrite Particles for Magnetic Resonance Imaging Thermometry”, J. Magn. Magn. Mater., 469, 550-557 (2019), (iv) Development of Ferrite-Based Temperature Sensors for Magnetic Resonance Imaging: A Study of Cu1-xZnxFe2O4, Physical Review Applied, 9, 054030 (2018), (v) “Zinc doped copper ferrite particles as temperature sensors for magnetic resonance imaging, AIP Advances 7, 056703 (2017), and (vi) “Ferromagnetic Particles as Magnetic Resonance Imaging Temperature Sensors”, Nature Communications, 7, Article number: 12415 (2016), all of which are incorporated herein by for what it teaches regarding magnetic particles that are useful in MRI thermometry.

In another more specific alternative embodiment, the temperature-stable hydrogen proton-rich material contains a polymer base material that is doped with magnetic particles (preferably 0.05 to 10 microns, more preferably about 3 microns, preferably a ceramic material containing oxides which contain iron, magnesium/yttrium, manganese and zinc, more preferably Cu_{0.24}Zn_{0.76}Fe₂O₄ or Mn_{0.48}Zn_{0.46}Fe_{2.06}O₄) at a concentration of about 0.05 mM to about 3 mM). Here the polymer base material (a) is mechanically stable from room temperature to ≤−65° C. or ≤−75° C.; (b) has no freezing transition in the temperature range of from about −75° C. to about +65° C., or about −50° C. to about +65° C.; (c) contains a high abundance of hydrogen protons to enable the magnetic resonance image to be detectable and brighter than the surrounding target; (d) has a narrow NMR linewidth and weak temperature dependence in the range of about 0° C. to about −50° C.; and (e) has a weak temperature dependence of the nuclear relaxation times T₁ and T₂. Here, the magnetic particles (a) cause a linewidth broadening with a strong temperature dependence; and (b) show a change in magnetization with a change in temperature over a temperature range of at least from about +37° C. to about −50° C. (or about −75° C. or about −65° C.) in the field of the MRI scanner.

In one embodiment, the temperature-stable proton-rich material is or contains a silicone polymer that is cured with temperature treatment, UV-treatment, or by the addition of a hardening component (e.g., silicone elastomers). In a preferred embodiment, the silicone polymer is allowed by regulatory authorities for long-term use in patients (e.g., silicones for contact lenses, breast implants, medical prosthetics, and the like). In one embodiment, the polymer is a silicone elastomer containing a hydride- and a vinyl-functional siloxane polymer that is reacted in the presence of a platinum complex catalyst, creating an ethyl bridge between the two reactive groups (such as e.g., DRAGON SKIN™ FX platinum cured silicone rubber, Smooth-On, Inc., Macungie, Pa.). In one embodiment, the polymer is a two-part cured polydimethylsiloxane gel (such as e.g., SYLGARD™ 527 silicone dielectric gel, Dow Chemical Company, Midland, Mich.), or a two-part cured polydimethylsiloxane elastomer (such as e.g., SYLGARD™ 184 silicone elastomer, Dow Chemical Company, Midland, Mich.).

In another embodiment, the polymer is a polyepoxide. Preferably, the polyepoxide contains phenyl and/or alkyl groups (e.g., methyl, ethyl, propyl or the like), which confer temperature-dependent linewidths to the polymer. (See, e.g., Contreras et al., Magnetic Resonance in Chemistry, 56:1158, 5 Jul. 2018.) For example, bisphenol-based epoxy resins contain both phenyl and methyl groups associated with the monomer subunits. In one embodiment, the polyepoxides are crosslinked by the addition of a curing agent to form a gel or rigid polymer. It is known in the art that pre-cured and cured polyepoxides demonstrate temperature-dependent changes in T₁ and T₂. (See, e.g., Kimoto et al., Analytical Sciences 24:915-920, 2008.)

In one embodiment, the temperature-stable hydrogen proton-rich material is shaped into an object having an aspect ratio ≥1, ≥2, or ≥10, such as a filament, and sized to fit within the target. For example, a 3 cm tumor may contain an object with a length that is about 3 cm±50%, i.e., 1.5 cm-4.5 cm. In one embodiment, the object is at least as long as an ice ball that forms within the target. In one embodiment, the target contains at least one object. In another embodiment, the target contains at least two objects. In one embodiment, at least one object is placed at or near the top of the ice ball, and another object is placed at or near the bottom of the ice ball. In one specific embodiment, trocar fibers, which are commonly used to mark the edges of a tumor in MRI guided surgeries, are replaced with filaments of the polymer material so as to provide both marking of the edge of the tumor and temperature information.

In one embodiment, the object is a temperature-stable proton-rich material coating on a hypodermic needle or probe that is inserted into the target (e.g., tumor). In another embodiment, the object is a hypodermic needle or probe made of the temperature-stable hydrogen proton-rich material and which is inserted into the target (e.g., tumor).

In one embodiment, the temperature of the polymer object is determined by measuring the brightness of T₁-weighted MM images. In one embodiment, the temperature of the polymer object is determined by measuring the brightness of T₂-weighted MM images. In one embodiment, the temperature of the polymer object is determined by measuring the brightness of T₂*-weighted MRI images.

In one embodiment, any one or more parameters in the MRI sequence are adjusted to increase the temperature sensitivity of the MM for a particular temperature-stable hydrogen proton-rich material, target body, and/or temperature. The MRI sequence parameters include flip angle, repetition time, and/or echo time. For example, for T₁ weighted images, changing the flip angle and/or changing the repetition time significantly improves the temperature sensitivity. For example, for T₂ weighted images, changing the flip angle and/or changing the echo time significantly improves the temperature sensitivity.

In one embodiment, the temperature of the polymer object is not determined by using a proton resonance frequency-based technique.

In a second aspect, the invention provides a method for determining the temperature of a target body by (a) placing a temperature-stable hydrogen proton-rich material, such as, e.g., a polymer, into or proximate to a target body, (b) heating the target body to a temperature >+40° C., (c) and imaging the a temperature-stable hydrogen proton-rich material using MRI, such that the level of brightness of the a temperature-stable hydrogen proton-rich material within the target body correlates with the temperature of the target body.

In one embodiment, the target body is a tissue within a patient. In a preferred embodiment, the tissue is a disease tissue including inter alia tumor, dysplasia, neoplasia, hyperplasia, carcinoma, sarcoma, and conducting tissue such as e.g., conductance cells at or near the atrioventricular node.

In one embodiment, the target body is heated to ≥+41° C., ≥+45° C., ≥+50° C., ≥+60° C., about +50° C., about +60° C., about +70° C., about +80° C., about +90° C., about +100° C., about +110° C., between +40° C. and +110° C., or between +50° C. and +150° C. In one embodiment, the temperature is a temperature that kills target cells. In one embodiment, the temperature is a temperature that kills target tumor cells and does not kill normal cells proximate to the tumor cells.

In one embodiment, the temperature-stable hydrogen proton-rich material is a polymer, more preferably a biocompatible polymer. In one embodiment, the temperature-stable hydrogen proton-rich material is mechanically stable within the range of about +15° C. to about +150° C. or within the range of about +30° C. to about +100° C. In one embodiment, the temperature-stable hydrogen proton-rich material has no freezing transition in the temperature range of from about 0° C. to about +80° C., about +10° C. to about +150° C., or about 0° C. to about +150° C. In one embodiment, the temperature-stable hydrogen proton-rich material contains a high abundance of hydrogen protons to enable the magnetic resonance image to be detectable and brighter than the surrounding target. In one embodiment, the temperature-stable hydrogen proton-rich material has strong and regular/monotonic temperature dependence of the nuclear relaxation times T₁, T₂, or T₂*, and the nuclear relaxation times T₁, T₂, or T₂* are in the range of about 5 ms to about 1,500 ms over the entire temperature range.

In a more specific embodiment, the temperature-stable hydrogen proton-rich material (a) is mechanically stable from about +15° C. to about +150° C.; (b) has no freezing transition within the temperature range of from about +10° C. to about +150° C.; (c) contains a high abundance of hydrogen protons to enable the magnetic resonance image to be detectable; (d) has strong and regular/monotonic temperature dependence of the nuclear relaxation times T₁, or T₂, or T₂*; and (e) has nuclear relaxation times T₁, T₂, or T₂* in the range of about 5 ms to about 1,500 ms over the entire temperature range.

In one embodiment, the temperature-stable proton-rich material is or contains a silicone polymer that is cured with temperature treatment, UV-treatment, or by the addition of a hardening component (e.g., silicone elastomers). In a preferred embodiment, the silicone polymer is allowed by regulatory authorities for long-term use in patients (e.g., silicones for contact lenses, breast implants, medical prosthetics, and the like). In one embodiment, the polymer is a silicone elastomer containing a hydride- and a vinyl-functional siloxane polymer that is reacted in the presence of a platinum complex catalyst, creating an ethyl bridge between the two reactive groups (such as e.g., DRAGON SKIN™ FX platinum cured silicone rubber, Smooth-On, Inc., Macungie, Pa.). In one embodiment, the polymer is a two-part cured polydimethylsiloxane gel (such as e.g., SYLGARD™ 527 silicone dielectric gel, Dow Chemical Company, Midland, Mich.), or a two-part cured polydimethylsiloxane elastomer (such as e.g., SYLGARD™ 184 silicone elastomer, Dow Chemical Company, Midland, Mich.).

In another embodiment, the polymer is a polyepoxide. Preferably, the polyepoxide contains phenyl and/or alkyl groups (e.g., methyl, ethyl, propyl or the like), which confer temperature-dependent linewidths to the polymer. (See, e.g., Contreras et al., Magnetic Resonance in Chemistry, 56:1158, 5 Jul. 2018.) For example, bisphenol-based epoxy resins contain both phenyl and methyl groups associated with the monomer subunits. In one embodiment, the polyepoxides are crosslinked by the addition of a curing agent to form a gel or rigid polymer. It is known in the art that pre-cured and cured polyepoxides demonstrate temperature-dependent changes in T₁ and T₂. (See, e.g., Kimoto et al., Analytical Sciences 24:915-920, 2008.)

In one embodiment, the temperature-stable hydrogen proton-rich material is shaped into an object having an aspect ratio ≥1, ≥2, or ≥10, such as a filament, and sized to fit within the target. For example, a 3 cm tumor may contain an object with a length that is about 3 cm±50%, i.e., 1.5 cm-4.5 cm. In one embodiment, the object is at least as long as an ice ball that forms within the target. In one embodiment, the target contains at least one object. In another embodiment, the target contains at least two objects. In one embodiment, at least one object is placed at or near the top of the ice ball, and another object is placed at or near the bottom of the ice ball. In one specific embodiment, trocar fibers, which are commonly used to mark the edges of a tumor in MRI guided surgeries, are replaced with filaments of the polymer material so as to provide both marking of the edge of the tumor and temperature information.

In one embodiment, the object is a temperature-stable proton-rich material coating on a hypodermic needle or probe that is inserted into the target (e.g., tumor). In another embodiment, the object is a hypodermic needle or probe made of the temperature-stable hydrogen proton-rich material and which is inserted into the target (e.g., tumor).

In one embodiment, the temperature of the polymer object is determined by measuring the brightness of T₁-weighted MM images. In one embodiment, the temperature of the polymer object is determined by measuring the brightness of T₂-weighted MM images. In one embodiment, the temperature of the polymer object is determined by measuring the brightness of T₂*-weighted MRI images.

In one embodiment, any one or more parameters in the MRI sequence are adjusted to increase the temperature sensitivity of the MM for a particular temperature-stable hydrogen proton-rich material, target body, and/or temperature. The MRI sequence parameters include flip angle, repetition time, and/or echo time. For example, for T₁ weighted images, changing the flip angle and/or changing the repetition time significantly improves the temperature sensitivity. For example, for T₂ weighted images, changing the flip angle and/or changing the echo time significantly improves the temperature sensitivity.

In one embodiment, the temperature of the polymer object is not determined by using a proton resonance frequency shift based technique.

In a third aspect, the invention provides an improved method for killing by hypothermal treatment problematic cells/tissue, such as atrioventricular cells associated with arrhythmia or hyperplastic cells, preferably tumor, cancer, or sarcoma cells. The improvement involves monitoring the temperature of the tissue that is being killed or removed to enable greater precision, efficacy, and safety in the procedure by better controlling time and space of reduced temperature. In one embodiment, the method includes the steps of placing one or more hydrogen proton-rich filaments into tissue comprising the problematic cells (e.g., tumor), placing a cryoablation probe into the tissue comprising the problematic cells, freezing the tissue comprising the problematic cells by injecting gas at high pressure within the probe to achieve a problematic cell-killing temperature (preferably ≤−50° C.), determining the temperature of the tissue comprising the problematic cells by MM imaging the one or more filaments by T₁, T₂, or T₂* weighted MR images, and then thawing the tissue comprising the problematic cells. The freeze-thaw cycle is repeated one or more times to effect killing of the problematic cells. Here, the brightness of the MRI-generated image of filaments correlates with the temperature of the tissue comprising the problematic cells and facilitates knowing when and where the killing temperature is achieved within the tissue comprising the problematic cells. Here also, the hydrogen proton-rich filaments, which are sized to fit within a tissue comprising the problematic cells (e.g., tumor) and placed within the tissue comprising the problematic cells to span an ice ball that forms within the problematic tissue, contain a hydrogen proton-rich material that is mechanically stable from room temperature to −65° C., and that enables the MRI image to be detectable and brighter than the surrounding target tissue. In one embodiment, the temperature is monitored during the thawing process.

In one embodiment, the hydrogen proton-rich material contains or consists of a polymer with monotonic temperature dependence of nuclear relaxation times T₁, T₂, or T₂* in the range of about 5 ms to about 1,500 ms over a range of temperatures to which the tissue (e.g., tumor) is subjected. In one embodiment, the polymer is a silicone elastomer or other biocompatible polymer. In another embodiment, the polymer is a polyepoxide or other biocompatible polymer.

In another embodiment, the hydrogen proton-rich material contains a polymer having a narrow NMR linewidth and weak temperature dependence in the range of about 0° C. to about −50° C. Here, to convey temperature-dependent nuclear resonance that is imageable across the cryoablation temperatures, the polymer is doped with a concentration of about 0.05 mM to about 3 mM of magnetic particles (the particles should have 0.003-10 microns average diameter) that contain or consist of oxides of zinc, and iron, such as, e.g., Cu_{0.24}Zn_{0.76}Fe₂O₄ or Mn_{0.48}Zn_{0.46}Fe_{2.06}O₄, with a diameter of ranging from 3 nm to 10 microns.

In a fourth aspect, the invention provides an improved method for killing by hyperthermal treatment problematic cells, such as atrioventricular cells associated with arrhythmia or hyperplastic cells, preferably tumor, cancer, or sarcoma cells. The improvement involves monitoring the temperature of the tissue that is being killed or removed to enable greater precision, efficacy, and safety in the procedure by better controlling time and space of increased temperature. An important aspect of killing tumors at high temperature is that the time necessary to kill a tumor depends on the temperature. For example, heating a portion of a tumor to 45° C. generally requires over an hour to kill that tissue. In contrast, heating a portion of a tumor to 60° C. requires less than a minute for killing the tissue (see, e.g., Sapareto and Dewey, Int J Radiat Oncol Biol Phys, 10(6):787-800 (1984)). By enabling the accurate monitoring of time at temperature, this improvement has a significant impact on a potential reduction of surgical times and reduction of recurrence of the cancer. In addition, at these high temperatures, it is important to monitor and thereby prevent or reduce any unintended damage to surrounding healthy tissue.

In one embodiment, the method includes the steps of placing one or more hydrogen proton-rich filaments into tissue comprising the problematic cells (e.g., tumor), placing an energy-delivering probe into the problematic tissue, heating the problematic tissue to achieve a problematic cell-killing temperature (preferably ≥+40° C. and ≤+150° C., or ≥+30° C. and ≤+80° C.), determining the temperature of the problematic tissue and/or healthy proximate tissue by MRI imaging one or more filaments by T₁, T₂, or T₂* weighted MR images. Here, the brightness of the MRI-generated image of filaments correlates with the temperature of the target tissue and facilitates knowing when and where the killing temperature is achieved within the problematic tissue. Here also, the hydrogen proton-rich filaments, which are sized to fit within the problematic tissue (e.g., tumor) and placed within the problematic tissue to span at least the killing area of the problematic tissue, contain a hydrogen proton-rich material that is mechanically stable from at least room temperature to about +80° C., about +100° C., or about +150° C., and that enables the MRI image to be detectable and brighter than the surrounding target tissue. In one embodiment, the temperature of the proximate healthy tissue is monitored.

In some embodiments, the energy-delivering probe delivers infrared radiation, microwave radiation, radio wave radiation, or laser radiation to the target object. In one embodiment, the problematic tissue (e.g., tumor) could be heated by introducing a thin glass fiber connected to a high-intensity laser (laser ablation), for example, with a diffuser at the end. Here, the glass fiber is positioned by MRI and, again, creates a nonuniform temperature distribution. In one embodiment, the glass fiber is coated with the hydrogen proton-rich material.

In another embodiment, the energy is delivered as radio-frequency electromagnetic waves impinging on the tumor or acting on particles embedded in the tumor and transduced into heat sufficient to kill the problematic tissue. In one embodiment, the hydrogen proton-rich material contains or consists of a polymer with monotonic temperature dependence of nuclear relaxation times T₁, T₂, or T₂* in the range of about 5 ms to about 1,500 ms over a range of temperatures to which the tumor is subjected. In one embodiment, the polymer is a silicone elastomer or other biocompatible polymer. In another embodiment, the polymer is a polyepoxide or other biocompatible polymer.

In one embodiment, any one or more parameters in the MRI sequence are adjusted to increase the temperature sensitivity of the MRI for a particular temperature-stable hydrogen proton-rich material, target body, and/or temperature. The MRI sequence parameters include flip angle, repetition time, and/or echo time. For example, for T₁ weighted images, changing the flip angle and/or changing the repetition time significantly improves the temperature sensitivity. For example, for T₂ weighted images, changing the flip angle and/or changing the echo time significantly improves the temperature sensitivity.

In another embodiment, the hydrogen proton-rich material contains a polymer that is doped with a concentration of about 0.05 mM to about 3 mM of magnetic particles (typical particle diameter 0.003-10 microns) that contain or consist of oxides of zinc, and iron, such as, e.g., Cu_{0.24}Zn_{0.76}Fe₂O₄ or Mn_{0.48}Zn_{0.46}Fe_{2.06}O₄, with a diameter of ranging from 3 nm to 10 microns.

In a fifth aspect, the invention provides a system for using MRI to determine the temperature of an object having a temperature ranging from ≤−65° C. to ≥+150° C. or from about −65° C. to about +80° C. In those embodiments in which the object is a diseased tissue (e.g., tumor or other undesirable tissue) that is subjected to cryoablation, the temperature range includes those temperatures necessary to effect tumor cell killing (or the killing of any problematic tissue that needs to be eliminated from a patient), such as, e.g., about −50° C., about −65° C., about −75° C. In those embodiments in which the object is a diseased tissue (e.g., tumor or other undesirable tissue) that is subjected to thermal ablation, the temperature range includes those temperatures necessary to effect tumor (or other) cell killing, such as, e.g., greater than about +30° C. to about +80° C. or above.

However, it is to be understood that the application of the subject system is not constrained to medical or surgical applications but is applicable to any target object that requires temperature control, especially at relatively extreme hot and/or cold temperatures.

In one embodiment, the system includes (a) a MM scanner, and (b) one or more hydrogen proton-rich filaments. In one embodiment, the MM scanner contains a 3 Tesla magnet. In one embodiment, the hydrogen proton-rich filament(s) contains or consists of a polymer having a strong and monotonic temperature dependent nuclear relaxation times at temperatures at least between 0° C. and −65° C., at least between −65° C. and +100° C., or at least between +30° C. and +80° C. In one embodiment, the polymer has monotonic temperature dependence of nuclear relaxation times T₁, T₂, or T₂* in the range of about 5 ms to about 1,500 ms over a range of temperatures, such as, e.g., about 0° C. to about −65° C., about −65° C. to about +100° C., or about +30° C. to about +150° C., or about +30° C. to about +80° C.

In one embodiment, the polymer is a silicone elastomer. In another embodiment, the polymer is a biocompatible silicone elastomer. In another embodiment, the polymer is a polyepoxide. In another embodiment, the polymer is a biocompatible polyepoxide. In yet other embodiments, the polymer may contain two or more polymers, such as a silicone elastomer and a polyepoxide, polymers with multiple different monomers, and the like.

In another embodiment, the hydrogen proton-rich filaments contain a polymer having a narrow nuclear magnetic linewidth with weak temperature dependent nuclear relaxation times at temperatures between 0° C. or +37° C. and −65° C. or between +30° C. and +80° C. or +110° C. In one embodiment, this polymer is doped with magnetic particles that show a change in magnetization with a change in temperature over a temperature range of at least from about +37° C. to about −65° C. or at least from about +30° C. to about +80° C. or about +110° C. while under the magnetic field. In one embodiment, the magnetic particles have an average diameter of about 0.01 to about 10 microns. In one embodiment, each magnetic particle contains zinc, and iron oxides, preferably as a ceramic or ceramic-like material, more preferably a Cu_{0.24}Zn_{0.76}Fe₂O₄ or a Mn_{0.48}Zn_{0.46}Fe_{2.06}O₄ material. In one embodiment, the polymer contains magnetic particles at a concentration of about 0.05 mM to about 3 mM within the polymer.

In one embodiment, the system also includes a cryoablation probe and a pressurized gas apparatus to enable freezing the tumor via the Joule-Thomson effect. In one embodiment, the object of which the temperature is determined is a tumor, such as a tumor undergoing cryoablation. In one embodiment, the tumor is within a patient. In another embodiment, the object of which the temperature is determined contains or is atrio-ventricular (AV) cells within a patient.

In one embodiment, the cryoablation probe (which can also be a hypodermic needle) is coated with the hydrogen proton-rich material. In another embodiment, the cryoablation probe is made in part of the hydrogen proton-rich material.

In one embodiment, the system also includes a probe to deliver energy to the target object. In one embodiment, the object of which the temperature is determined is a tumor, such as a tumor undergoing hyperthermal ablation. In one embodiment, the tumor is within a patient. In another embodiment, the object of which the temperature is determined contains or is atrio-ventricular (AV) cells within a patient.

In one embodiment, the energy-delivery probe is coated with the hydrogen proton-rich material. In another embodiment, the energy-delivery probe is made in part of the hydrogen proton-rich material. In a specific embodiment, the energy-delivery probe is an optical fiber that delivers high energy laser radiation. In one embodiment, the optical fiber is coated with the hydrogen proton-rich material to produce a dielectric waveguide with MRI-temperature indication properties.

### BRIEF DESCRIPTION OF THE DRAWINGS

FIG. 1 is a transmission electron micrograph of PEG coated Mn_{0.48}Zn_{0.46}Fe_{2.06}O₄ particles. Mean value of size is 7.8 nm with a standard deviation of 2.2 nm.

FIG. 2 is a line graph depicting the temperature dependence of magnetization in Mn_{0.48}Zn_{0.46}Fe_{2.06}O₄. X-axis shows temperature in degrees Kelvin. Y-axis shows mass magnetization in ampere times square meter per kilogram. Note the rapid, nearly linear, decrease of magnetization with temperature.

FIG. 3 is a line graph depicting signal strength as a function of temperature and flip angle (α). Black line represents α=20°; red line represents α=30°; blue line represents α=40°; green line represents α=50°; gold line represents α=60° light blue line represents α=70°; magenta line represents α=80°; and violet line represents α=90°.

FIG. 4 is a dot plot depicting spin-spin T₂ nuclear relaxation time in milliseconds as a function of temperature in degrees Celsius in platinum cured soft silicone rubber compounds Ecoflex 00-20 and Ecoflex 00-30 materials. The temperature range is −60° C. to +60° C.

FIG. 5 is a diagram depicting a cryoablation method showing the tumor, ice ball, and cryoablation probe (labeled needle applicator) for injection of cooling gas (argon) and warming gas (helium).

FIG. 6 is a diagram depicting a tumor, an internal ice ball, and two hydrogen proton-rich polymer filaments, where dark regions within each filament indicate lower temperatures. Different embodiments allow brighter regions to indicate lower temperatures.

FIG. 7 is a dot plot depicting T₁ nuclear relaxation time (y-axis in milliseconds) in various silicone polymer formulations relative to temperature (x-axis in degrees Celsius). Open circles (∘) represent 0.59 Tesla NMR data obtained from Ecoflex 00-20 polymer). Open squares (□) represent 0.59 Tesla NMR data obtained from Ecoflex 00-30 polymer. Open triangles (Δ) represent 3 Tesla NMR data obtained from Dragon Skin™ silicone. Open diamonds (⋄) represent 3 Tesla MRI data obtained from Dragon Skin™ silicone.

FIG. 8 is a line graph depicting magnetic resonance image (MRI) intensity (y-axis in arbitrary units) relative to temperature (x-axis in degrees Celsius) of T₁-weighted gradient echo images in Dragon Skin™ FX silicone.

FIG. 9 is a MR image of a Dragon Skin™ polymer phantom taken at −40° C. (left panel) and +20° C. (right panel). The phantom at a higher temperature is clearly darker. Note, the central region was doped with magnetic particles and is not visible in this T₁ weighted image.

FIG. 10 is a dot plot depicting brightness (image intensity y-axis) as a function of temperature (x-axis) for different regions of a Dragon Skin™ siloxane elastomer phantom MR image. The inset depicts the MM image and the specific regions of interest numbered 1-4 from which the plotted data was obtained. Open circles (∘) represent data obtained from region of interest 1. Open squares (□) represent the average of the data obtained from regions of interest 1-4.

FIG. 11 is a magnetic resonance image of a polymer phantom (Ecoflex 00-30) with a temperature gradient between the center and outer edge. The brightness of the image correlates to temperature, as directly measured via two thermocouples.

FIG. 12 is a dot plot showing magnetic resonance image brightness as a function of temperature for silicone polymers Ecoflex 00-30 and Ecoflex 00-20 at three positions along the radius of the phantom. Green dots represent a position 3 mm from center; blue dots represent a position 9 mm from center; and red dots represent a position 13 mm from center.

FIG. 13 is a dot plot showing magnetic resonance image brightness (y-axis) as a function of temperature (y′-axis, right side) for different locations (x-axis) acquired during phantom cooling. L indicates an effective typical spatial resolution defined as the distance at which one can identify a temperature change of about 3 degrees. The inset is an MRI showing the region from which the data is sampled.

FIG. 14 is a false color MM temperature map of an Ecoflex 00-30 silicone polymer phantom with a temperature gradient between the center and outer edge. This represents a smoothed version of the image in FIG. 11 after removal of the Gibb's artifact ringing.

FIG. 15 shows imaging and graphical depictions of MRI thermometry of a magnetic ferrite particle-embedded polymer phantom.

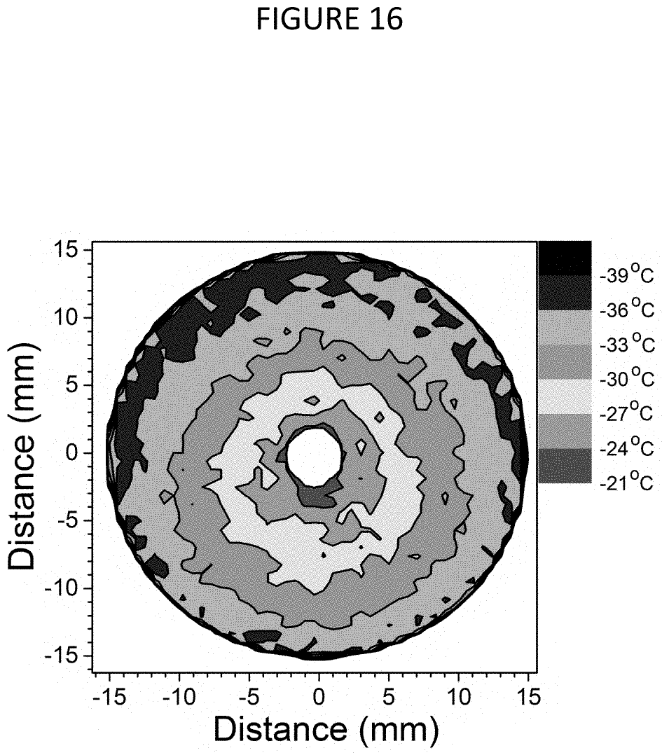

The image within FIG. 15 labeled as (a) depicts a MR sagittal scout image of the phantom with axial slice indicated by the parallel lines. The dark vertical region in the center comes from the glass fiber temperature sensor.

The image within FIG. 15 labeled as (b) depicts a T₂-weighted axial slice showing four glass vials (each 5 mm in diameter) filled with different concentrations of 3-micron sized Cu_{0.24}Zn_{0.76}Fe₂O₄ particles. Vials are inserted in the silicone. The surrounding temperature is about +20° C.

The image within FIG. 15 labeled as (c) depicts a T₂-weighted axial slice showing four glass vials (each 5 mm in diameter) filled with different concentrations of 3-micron sized Cu_{0.24}Zn_{0.76}Fe₂O₄ particles. Vials are inserted in the silicone. The surrounding temperature is about −40° C.

The graph within FIG. 15 labeled as (d) is a dot plot depicting magnetic resonance image brightness (y-axis in arbitrary units) as a function of temperature (x-axis in degrees Celsius). Note that lower temperatures are darker. The concentration of Cu_{0.24}Zn_{0.76}Fe₂O₄ was 0.5 mM.

FIG. 16 shows a false color MR image of Ecoflex 00-30 silicone polymer with the different color regions spanning three degrees Celsius.

### DETAILED DESCRIPTION OF SPECIFIC EMBODIMENTS

Before the present methods are described, it is to be understood that this invention is not limited to particular methods or systems, and experimental conditions described, as such methods or systems and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means±5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

The system and method of the invention provides substantial advantages over current methods for measuring the temperature of very cold or very hot objects by magnetic resonance imaging. For example, it is problematic in the art for accurately measuring and mapping the temperature of frozen objects by magnetic resonance imaging due to problems associated with linewidth broadening and concomitant loss of image brightness.

### Proton-Rich Temperature Indicators

Disclosed are systems and methods for overcoming this problem. In a specific embodiment, the temperature of an object (the first object) is determined by placing a hydrogen proton-rich polymer object (the second object) into the first object and then imaging the second object by magnetic resonance imaging (MRI) to produce an image that changes brightness with changing temperature.

Multiple materials were identified that show significant changes in their MRI brightness with temperature. These materials, some of which are already approved for use in the human body by the Food and Drug Administration, are biocompatible and nontoxic.

These materials include low temperature stable polymers such as, but not limited to silicone elastomers (Dow Sylgard silicon elastomers and gels), perfluoroalkoxy (PFA), polyimides (PI), ultra high molecular weight polyethylene (UHMW-PE), polytetrafluoroethylene (PTFE), polychlorotrifluoroethylene (PCTFE), fluorinated ethylene propylene (FEP), polyvinyl fluoride (PVF), polyvinylidene fluoride (PVDF), polyethylene terephthalate (PET), and the like.

Primary criteria for the preferred materials which will work at low temperature include the following attributes. (1) There is no freezing transition of the material within temperature range from about −65° C. to about +65° C., which means that the material is mechanically stable within this temperature range. (2) The material contains a high abundance of hydrogen protons, as occurs in many plastics and polymers. This allows the MM signal to be easily detectable, substantially brighter than the surrounding tissue, and clearly visible. (3) The material exhibits strong and regular/monotonic temperature dependence of the nuclear relaxation times T₁ and/or T₂. This allows one to see a change in MRI brightness as the temperature changes. (4) The material exhibits nuclear relaxation times T₁ and T₂ in the range of about 5 to about 1,000 or 1,500 ms over the entire temperature range.

A number of preferred materials that fulfill these requirements fall into the category of silicone polymers (silicone rubbers, elastomers) that can be cured by temperature or by adding a hardening component, such as platinum- or palladium-based catalysts. Examples of these materials can be found in Patterson, R. F., in Handbook of Thermoset Plastics (Second Edition), 1998. DOI: 10.1016/B978-081551421-3.50012-6. Some materials in this class of substances are already allowed for long-term use in bodies, e.g. breast implants and contact lenses. Similar materials are also used for medical prosthetic and cushioning applications.

Proton-rich materials (e.g., polymers) that are useful as low-temperature-indicating second objects can be selected using NMR. Although the ultimate objective is to find second object materials that have a significant change in MRI brightness as a function of temperature, NMR can be used to indicate MRI behavior.

Here, a basic quality for a useful second object is an NMR linewidth near the range of 100-1,000 Hz which varies smoothly with temperature. An important example of a material that doesn't work is water. The linewidth of deionized water, as it freezes, drastically increases with the lowering of temperature. This significant increase in linewidth is seen in an MM image as a black ice ball as the tissue freezes. The linewidth of biological tissue reaches 40 kHz near −50° C.

Dragon Skin™ FX, Smooth-On Platinum Cured Silicones Ecoflex 00-20 and Ecoflex 00-30 (Smooth-On, Inc., Macungie, Pa.), SYLGARD™ 527 silicone dielectric gel and SYLGARD 184 silicone elastomer (Dow Chemical Company, Midland, Mich.), were tested using a low-field pulsed NMR spectrometer for thermal changes of linewidth in temperature between −50° C. to +20° C. Dragon Skin™ FX showed a very strong signal from hydrogen protons. Above +30° C., the linewidth stays in range of 100 Hz, and increases to 300 Hz at −45° C., well below of 500 Hz, which is the top limit for MM usefulness. Similar behavior was found for the silicone elastomer 527.

Useful silicone materials typically have a low thermal conductivity to enable proper measurement of local temperature; a high thermal stability, which means that their chemical and physical properties change very little from −75° C. to +75° C.; a high chemical resistance to attack by oxygen and ozone; and preferably biocompatibility and no toxicity (see U. A. Daniels, Silicone Breast Implant Materials, Swiss Medical Weekly, Vol 142, (2012) doi:10.4414/smw.2012.13614).

Other useful polymers include Tygon™ tubing polymers (Saint-Gobain, La Défense, Courbevoie, France), such as, e.g., Tygon™ E1000 Lab Tubing, Tygon Medical/Surgical Tubing S-50-HL, Tygon Medical Tubing S-54-HL, Tygon 2275 High Purity Tubing, and other polyurethane, polyvinyl chloride, polyepoxide, and silicone polymers.

Proton-Rich Material Doped with Magnetic Particles

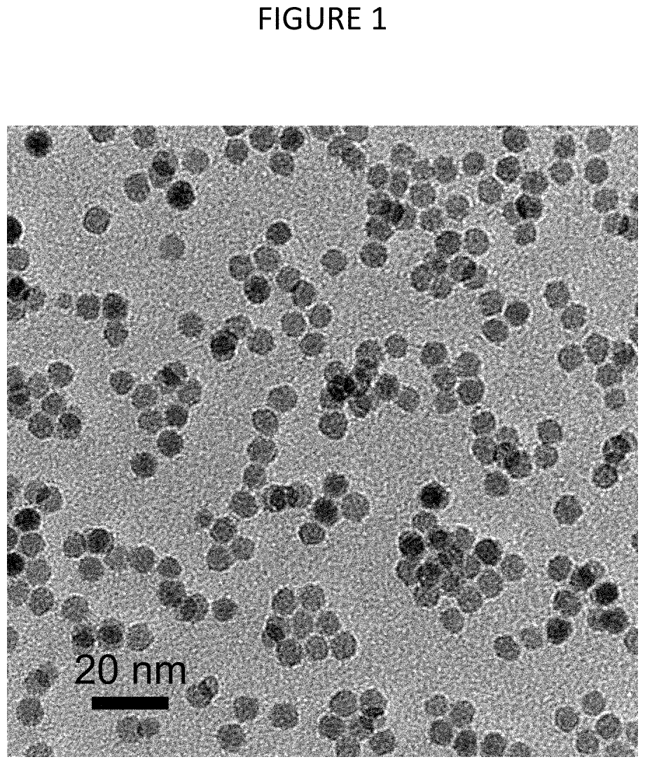

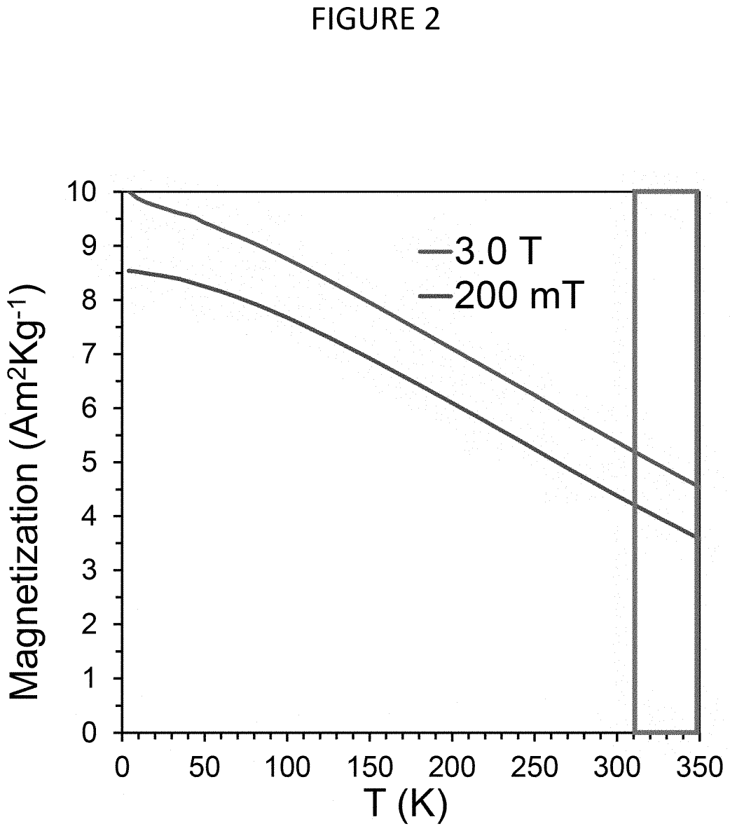

In an alternative embodiment, the second object is made from a polymer base material (e.g., silicones, epoxies, polyurethanes, PVCs, and the like) that is doped with magnetic particles. Here, the polymer base material has an initial narrow NMR linewidth and weak temperature dependence in the range of 0° C. to −50° C., but when doped with the magnetic material, has a strong temperature dependent linewidth. The criteria for materials in this embodiment, which will work at low temperatures, include the following attributes. (1) The material has mechanical stability from room temperature to at least −65° C. In other words, there is no freezing transition in this temperature range. (2) The material contains a high abundance of hydrogen protons to enable the MRI signal to be bright at higher temperatures and brighter than the surrounding tissue. (3) The base material should have an initial narrow NMR linewidth and weak temperature dependence in the range 0° C. to −50° C. After doping with designed magnetic particles, the linewidth will have a strong temperature dependence. The magnetic particles must be designed to have a significant change in magnetization with temperature over the range from body temperature to −50° C. in the magnetic field of the MRI scanner. For example, Cu_{0.24}Zn_{0.76}Fe₂O₄ particles with a diameter of 3 microns work well, but other materials with an appropriate variation of magnetization with temperature, such as, e.g., Mn_{0.48}Zn_{0.46}Fe_{2.06}O₄ particles (see FIGS. 1 and 2), will also work. The magnetic particles must have an appropriate concentration in the surrounding material. Often the appropriate range is 0.05 mM to 3 mM. (4) The base material should have a weak temperature dependence of the nuclear relaxation times T₁ and T₂.

In general, the magnetic particles useful in the practice of the invention can be of any material or combination of materials where the magnetization of the material changes substantially as a function of temperature, in the temperature range of interest and at the fields typical in MRI systems. This class of materials includes, but is not limited to, ferromagnets, ferrimagnets, paramagnets, and canted antiferromagnets among others. Some representative materials include Permalloy doped with Cu, Gd, Gd doped with Cu, FeBO₃, rare-earth doped iron-oxide garnets, and alloys of FeGd, Co/Gd, and the like. The exact compositions will depend on the desired temperature operational range and the applied magnetic field.

In some embodiments, the magnetic particles are iron oxides doped with one of more first d-block series (3d) transition metals, such as e.g., Zn, Cu, and Mn, and other divalent or trivalent metals, such as e.g., magnesium (Mg) or yttrium (Y).

In one embodiment, polymers are doped with about 0.1 mM to about 10 mM, about 0.1 mM, about 0.5 mM, about 1 mM, about 2 mM, about 3 mM, about 4 mM, about 5 mM, about 6 mM, about 7 mM, about 8 mM, about 9 mM, about 10 mM, or about 20 mM of magnetic particles. In one embodiment, certain polymers, such as e.g., Ecoflex silicones, Sylgard 527 dielectric gel, and the like, which show strong NMR signals in the range of ±60° C., can be doped with magnetic particles to control T₁, T₂, and T₂* values, leading to MR images which have a strong temperature dependent brightness.

### MRI Parameters

It is possible to optimize the MRI temperature-sensitivity of a given material. The temperature sensitivity can be optimized by adjusting one or more parameters of the MRI sequence used to measure temperature or temperature change. In one embodiment, the temperature-dependent change in MRI intensity can be significantly improved by varying the flip angle. In another embodiment, the temperature-dependent change in MRI intensity can be significantly improved by varying the echo time. In another embodiment, the temperature-dependent change in MRI intensity can be significantly improved by varying the repetition time. In other embodiments, the temperature-dependent change in MM intensity can be significantly improved by varying any one or more of the echo time, repetition time, and the flip angle.

In one embodiment, the repetition time (TR) and/or flip angle (a) is determined or selected according to Equation 1, where S represents the signal strength (intensity), TR is the repetition time, T₁ is a relaxation time and a is the flip angle.

$$S = \frac{sin ( α ) ( 1 - {e}^{- \frac{TR}{{T}_{1}}} )}{1 - cos ( α ) {e}^{- \frac{TR}{{T}_{1}}}} \qquad Eq . 1$$

Equation 1 is useful for obtaining T₁-weighted images. In some embodiments, a similar calculation can be done for T₂ or T₂*-weighted images.

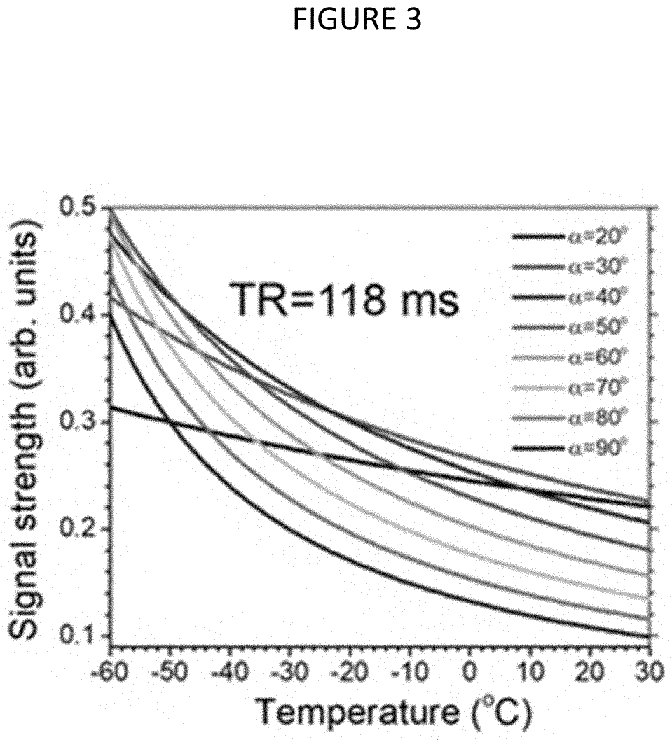

FIG. 3 presents the calculated S values, which are expected to be proportional to the MRI brightness, based on Equation 1, as a function of flip angle and temperature with a repetition time of 118 ms. The flip angle plays an important role in the MRI gradient echo signal amplitude and in the change in the MR image brightness with temperature. A larger flip angle generally produces bigger changes in the signal (thus larger differences in MR image brightness) as the temperature is changed, leading to an improved temperature resolution. Through these calculations, it is possible to optimize the MRI results to give an improved temperature resolution for a given material, temperature range, and MRI sequence.

In one embodiment, a flip angle is selected to provide a large change in signal strength (S) with temperature. In another embodiment, the repetition time is selected to provide a large change in signal strength with temperature. In another embodiment, echo time is selected to provide a large change in signal strength with temperature.

In one embodiment, the temperature sensitivity of the MRI signal obtained from the proton-rich material is increased by changing parameters in the MRI sequence. In particular, for T₁-weighted images, the flip angle and/or the repetition time is adjusted to increase the temperature sensitivity. For T₂-weighted images, the flip angle and/or the echo time is adjusted to increase the temperature sensitivity.

### Cryoablation Applications

In some embodiments, the first object is a tumor in a patient and the hydrogen proton-rich polymer object (the second object) is inserted into the tumor. Since the utility of the second object is to measure the temperature of the first object, the second object must be sized appropriately to cover the breadth and depth of the primary object. In some case, two (or more) second objects can be placed into the first object to enable broad coverage of temperature throughout the first object.

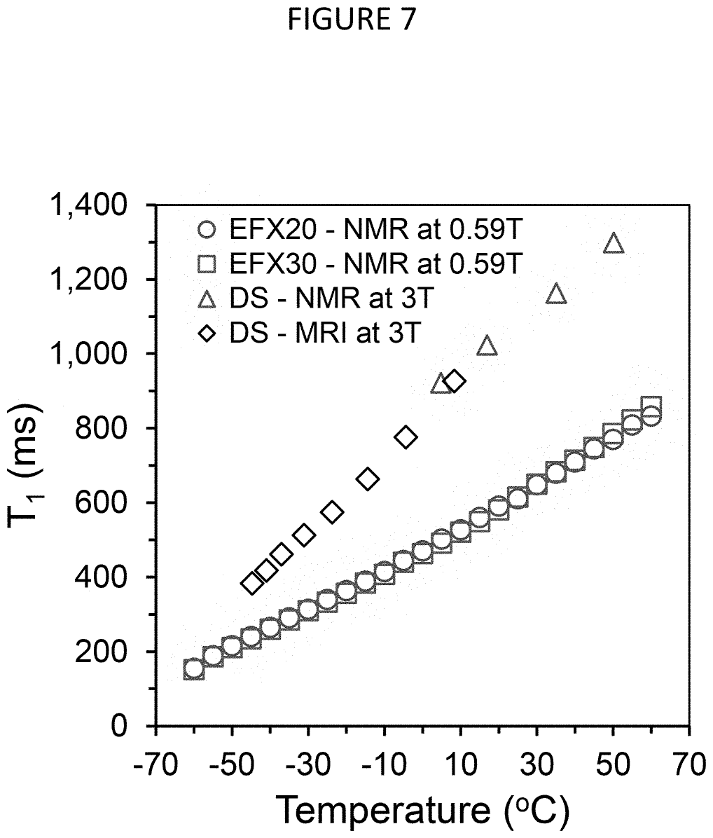

In the case of a tumor first object, the second object may be fashioned into a filament shape (i.e., having a relatively large aspect ratio) and inserted into the tumor. This scenario is depicted in FIG. 6, where two filaments are placed within the tumor.

Thus, in one embodiment, the second object has an aspect ratio that is ≥2, ≥4, ≥8, ≥16, ≥64, or ≥128, or between about 2 and about 50.

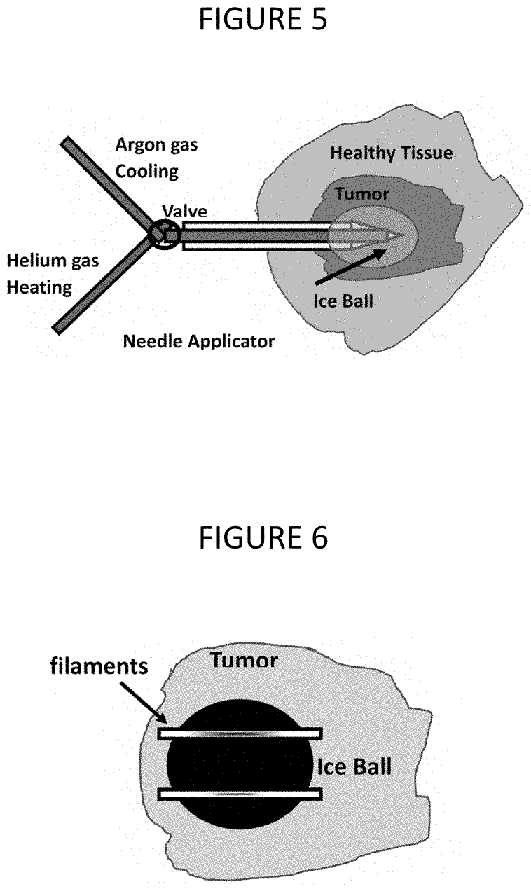

Magnetic Resonance Imaging (MM) is used to guide a variety of interventional cancer surgeries, resulting in less invasive procedures and significantly reduced side effects. Early techniques often killed tumors by heating them above 45° C., with heating provided by a laser beam guided into the tumor by a glass fiber and positioned by MRI [see Bomers, J. G. R., Cornel, E. B., Fütterer, J. J., Jenniskens, S. F. M., Schaafsma, H. E., Barentsz, J. O., Sedelaar, J. P. M., Hulsbergen-van de Kaa, Ch.A., and Witjes, J. A. (2017) MRI-guided focal laser ablation for prostate cancer followed by radical prostatectomy: correlation of treatment effects with imaging. World J. Urol. 35. 703-711. DOI: 10.1007/s00345-016-1924-1]. Recently there has been a move to killing tumors by freezing instead of by heating. MRI-guided cryoablation is an interventional procedure which kills tumors by freezing [see Morrison, P. R., Silverman, S. G., Tuncali, K., and Tatli S. (2008) MRI-guided cryotherapy. J Magn Reson Imaging. 27. 410-20. doi: https://doi.org/10.1002/jmri.21260; Babaian. R. J., Donnelly, B., Bahn, D., Baust, J. G., Dineen, M., Ellis, D., Katz, A. S., Pisters, L, Rukstalis, D., Shinohara, K., and Thrasher, J. B. (2008) Best Practice Statement on Cryosurgery for the Treatment of Localized Prostate Cancer. J. Urol. 180. 1993-2004. DOI: 10.1016/j.juro.2008.07.108; Barqawi, A. B., Huebner, E., Krughoff, K., and O'Donnell, C. I. (2018) Prospective outcome analysis of the safety and efficacy of partial and complete cryoablation in organ-confined prostate cancer. Urology 112. 126-31. doi: https://doi.org/10.1016/j.urology.2017.10.029; de Marini, P., Cazzato, R. L., Garnon, J., Shaygi, B., Koch, G., Auloge, P., Tricard, T., Lang, H., and Gangi, A. (2019) Percutaneous MR-guided prostate cancer cryoablation technical updates and literature review. British Institute of Radiolog. Open. 2019. 20180043. DOI: 10.1259/bjro.20180043; and Woodrum, D. A., Kawashima, A., Gorny, K. R., and Mynderse, L. A. (2017) Prostate cancer: state of the art imaging and focal treatment. Clinical Radiology. 72. 665-679. DOI: 10.1016/j.crad.2017.02.010]. Cryoablation locally freezes the tumor, creating an ice ball (see FIG. 5), and results in direct damage to tumor cells by a repeated process of freezing and thawing. Cells usually die at temperatures between −20° C. and −50° C. due to membrane ruptures, cellular dehydration and local ischemia [see Baust, J., Gage, A. A., Ma, H., and Zhang, C. M. (1997) Minimally invasive cryosurgery-technological advances. Cryobiology. 34. 373-384. DOI: 10.1006/cryo.1997.2017; and Tatli, S., Acar, M., Tuncali, K., Morrison, P. R., and Silverman, S. (2010) Percutaneous cryoablation techniques and clinical applications. Diagn. Interv. Radiol. 16. 90-95. DOI: 10.4261/1305-3825.DIR.1922-08.0]. The positioning of the needle applicator (a.k.a. cryoablation probe) is guided by MM.

MRI guided cryoablation provides multiple advantages such as reduced side effects, identification of the edges of the tumor, and localization of the ice ball. Unfortunately, below 0° C., standard MRI is unable to provide any actual image of the frozen tissue. Basically, the image of the ice ball simply turns black. In other words, the surgeon can know that there is an ice ball but has no information about the temperature inside the ice ball. In most cases, the temperature inside the ice ball is different near the applicator compared to the edge of the ice ball. For example, the edge of the ice ball may be at 0° C., while the inner portions of the ice ball are at colder temperatures. Since one must reach temperatures well below freezing to ensure the death of the tumor cells, this is a critical lack of information [see van den Bosch, M. A., Josan, S., Bouley, D. M., Chen, J., Gill, H., Rieke, V., Butts-Pauly, K., and Daniel, B. L. (2009) MM imaging-guided percutaneous cryoablation of the prostate in an animal model: in vivo imaging of cryoablation-induced tissue necrosis with immediate histopathologic correlation. J. Vasc. Interv. Radiol. 20. 252-258. DOI: 10.1016/j.jvir.2008.10.030; Josan, S., Bouley, D. M., van den Bosch, M., Daniel, B. L., and Butts Pauly, K. (2009) MM-guided cryoablation: in vivo assessment of focal canine prostate cryolesions. J. Magn. Reson. Imaging. 30. 169-176. DOI: 10.1002/jmri.21827; Woodrum, D. A., MD, Kawashima, A., Gorny, K. A., Lance, A., and Mynderse, L. A. (2019) Magnetic Resonance—Guided Prostate Ablation. Semin. Intervent. Radiol. 36. 351-366. DOI: 10.1055/s-0039-1697001].

The method commonly used to measure temperature in thermal ablations where the tumor is heated, proton resonance frequency (PRF) shift, is completely ineffective at low temperatures [see De Poorter, J., De Wagter, C., De Deene, Y., Thomsen, C., Stahlberg, F., and Achten, E. (1995) Noninvasive MRI thermometry with the proton resonance frequency (PRF) method: in vivo results in human muscle. Magn. Reson. Med. 33. 74-81. DOI: 10.1002/mrm.1910330111; Rieke, V., and Pauly, K. B. (2008) MR Thermometry. J. Magn. Reson. Imaging. 27. 376-390. DOI: 10.1002/jmri.21265; and H. Odéen, H., and Parker, D. L. (2019) Magnetic resonance thermometry and its biological applications Physical principles and practical considerations, Prog. Nucl. Reson. Spectrosc. 110, 34-61. DOI: 10.1016/j.pnmrs.2019.01.003]. This is caused by the large increase in linewidth for protons as the material freezes.

Another possibility, ultrashort echo-time MRI sequences, allows visualization of the temperature inside the ice ball, but only at temperatures above −40° C. with acquisition times of more than 1 minute, [see Overduin, C. G., Futterer, J. J., and Scheenen, T. W. (2016) 3D MR thermometry of frozen tissue: Feasibility and accuracy during cryoablation at 3T. J. Magn. Reson. Imaging. 44. 1572-1579. DOI: 10.1002/jmri.25301]. As a result, this method is also not useful in clinical settings. The lack of knowledge about the temperature in real time can produce multiple unwanted outcomes. These include: 1) The low temperature within the ice ball may not completely kill the tumor tissue, resulting in a recurrence of the cancer, or 2) To ensure appropriate temperature for killing the tumor and the lack of information about where this occurs, the ice ball must extend well beyond the tumor, damaging healthy tissue.

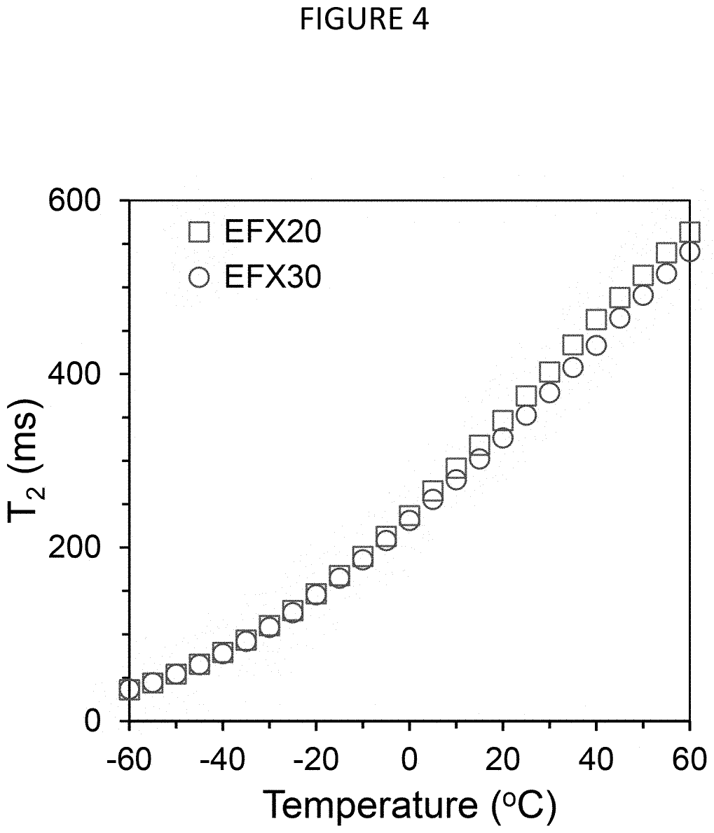

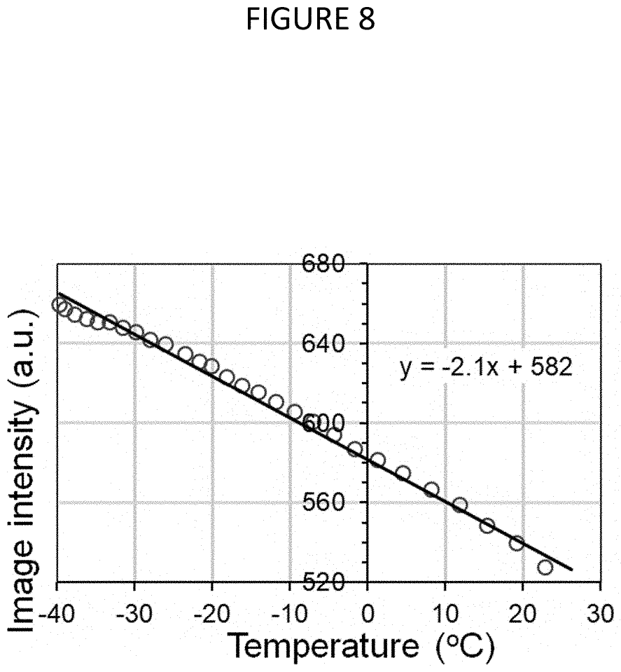

As described herein, certain classes of polymers have both T₁ and T₂ nuclear relaxation times that vary significantly with temperature, both above and below 0° C. See FIGS. 4 and 7. This property enables the creation of materials that show substantial variations in MM image brightness as a function of temperature. These materials can therefore be effectively used as local indicators of temperature. Filaments made of bio-compatible polymer materials will provide relevant information to allow the creation of 3D temperature maps during MRI-guided surgeries.

In one embodiment, the second object filament is made with a known and uniform doping of magnetic particles, such that each filament has a known brightness as a function of temperature. These filaments (as well as the non-doped filaments described above) are removed after the surgery, removing with them the magnetic particles. Another advantage of embedding magnetic particles in a polymer is that the particles are encased in a biocompatible, non-toxic, non-interacting substance, significantly reducing or eliminating the potential toxicity of the magnetic particles. Also, as pointed out above, MRI images of tissues are generally dark compared to the brightness of the filament. Thus, placing the particles in a filament with a brighter MRI signal provides a bright object with enhanced temperature resolution.

### Example 1: Temperature Measurement Across Tumor

Turning to FIG. 6, an ice ball having a temperature below freezing within a tumor appears black in MRI, but filaments, which are made of or containing an appropriate hydrogen proton-rich material, have a brightness that is temperature dependent at subzero temperatures. Here, the material (i.e., the filaments) is brighter at higher temperature and darker at lower temperature (in some cases depending on the material, the filaments may be brighter at lower temperatures), thereby providing a local measurement of temperature along the filament.

The placement of several filaments within the tumor enables the obtainment of information about temperature differences between the top and bottom of the ice ball. Based on the range of temperature along the entire length of each filament, one can mathematically create a map of the temperature throughout the ice-ball, superimposed on the image of the anatomical features. The filaments are removed after the surgery.

As described in the literature for photothermal ablation of prostate tumors (see Ardeshir R. Rastinehad, Harry Anastos, Ethan Wajswol, Jared S. Winoker, John P. Sfakianos, Sai K. Doppalapudi, Michael R. Carrick, Cynthia J. Knauer, Bachir Taouli, Sara C. Lewis, Ashutosh K. Tewari, Jon A. Schwartz, Steven E. Canfield, Arvin K. George, Jennifer L. West, and Naomi J. Halas, Gold nanoshell-localized photothermal ablation of prostate tumors in a clinical pilot device study, PNAS Sep. 10, 2019 116 (37) 18590-18596; https://doi.org/10.1073/pnas.1906929116), a useful distance between filaments is from about 5 mm to about 7 mm.

### Example 2: Temperature Dependent Materials

Dragon Skin™ FX (Smooth-On, Inc. Macungie, Pa.), silicone elastomers 527 and 184 (Sylgard, Dow, Midland, Mich.), and other polymers, including Smooth-On Platinum Cured Silicones Ecoflex 00-20 and Ecoflex 00-30 were tested for thermal changes of linewidth for temperatures between −50° C. to +50° C. using 3 Tesla pulsed NMR spectroscopy (3T NMR). As shown in FIG. 7, the Dragon Skin sample shows a strong temperature-dependent T₁ with a change of about 300% in the range −50° C. to +50° C. This linear change in T₁ with temperature was demonstrated to lead to a near linear change in MRI brightness with temperature as well. Similar behavior was found for the elastomers, Ecoflex 00-20 and Ecoflex 00-30 as shown in FIG. 7, Sylgard 527™, and some commercially available polymers with similar properties, including Tygon™ E1000 Lab Tubing (Saint-Gobain S.A., Courbevoie, France).

FIG. 7 depicts measurements of the T₁ nuclear relaxation time as a function of temperature for different materials. Dragon Skin (DS), Ecoflex 00-20, and Ecoflex 00-30 polydialkylsiloxanes were measured with both NMR and MRI at 3.0 T. NMR data were obtained with an inversion recovery method. The MM data were obtained with combined spin-echo and inversion recovery method. The T₁ time in pure Ecoflex 00-20 and Ecoflex 00-30 polymers were measured at 0.58 T using an NMR pulsed spectrometer. Note the change in relaxation from −65° C. to +65° C. is larger than 300% in some cases.

### Example 3: Magnetic Resonance Image Brightness of Materials

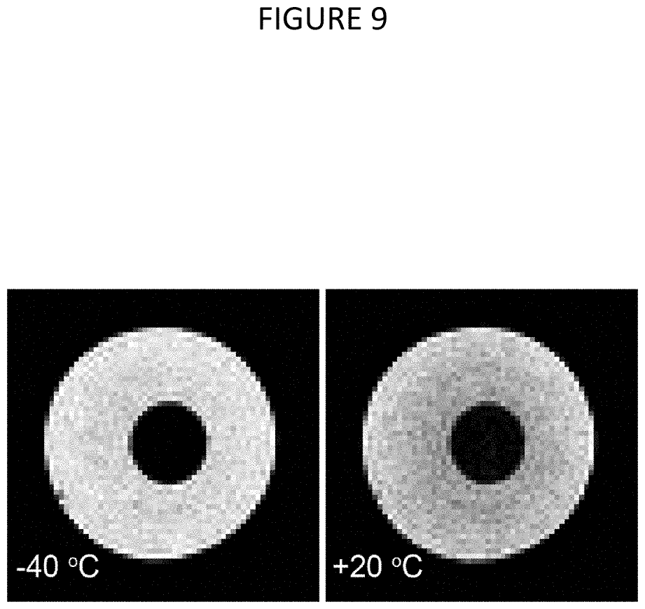

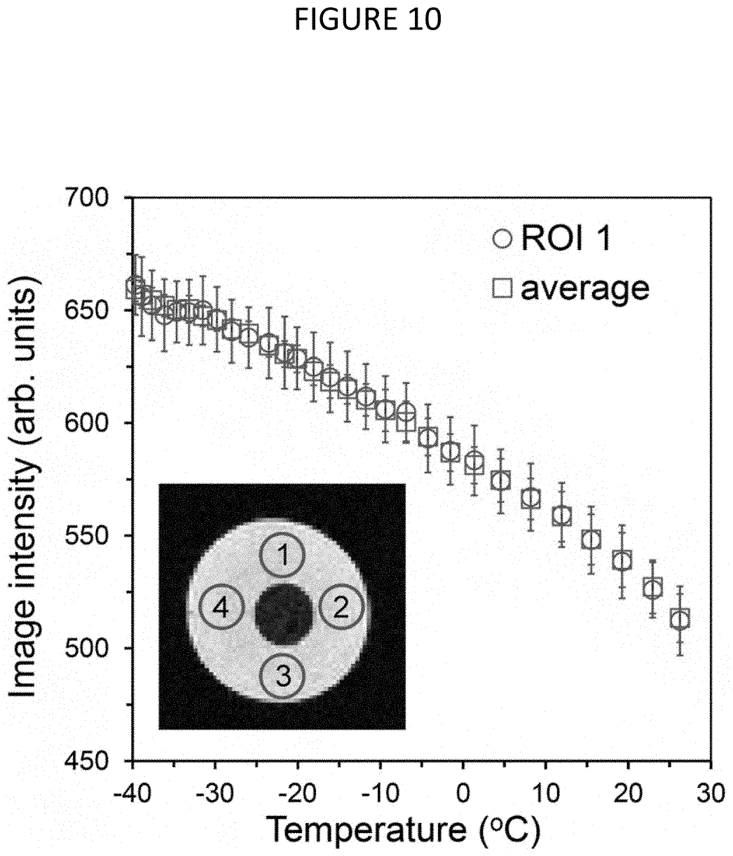

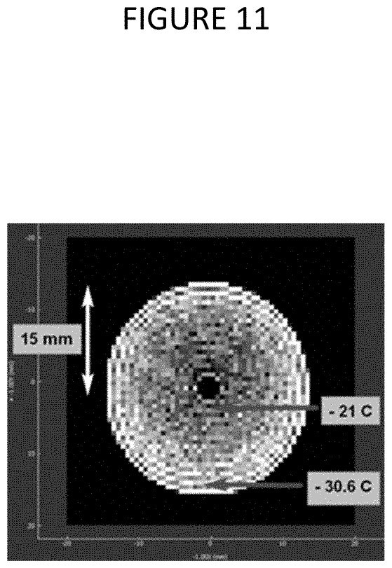

Phantoms of a variety of materials were made in cylindrical shape with a height of 2 inches and a diameter of 1 inch. Dry ice was packed around the material to establish the low temperature gradient and the phantom was insulated to maintain a slow cooling rate. FIG. 9 shows a cross-sectional MR image of such a phantom at two different temperatures. For data analysis, a small region of the phantom was selected because of the slow cooling rate and the need for close-to-uniform temperature across the region. MRI images were acquired. FIGS. 8 and 10 are for Dragon Skin™ polymers at different temperatures and brightness was correlated to temperature.

MRI image intensity versus temperature for the Dragon Skin material was measured in four different regions of the phantom (regions 1-4) and compared to the average intensity and variation over the entire MRI image slice of the phantom. As shown in FIG. 10, the results in region 1 accurately reflect the results averaged over the entire phantom. Regions 2, 3, and 4 each gave similar results. The error in the MRI intensity, averaged over the sample, is less than 2% of its value at low temperatures. This leads to a temperature resolution, for bulk materials, of about 5° C.

### Example 4: Measurement of Spatial Temperature Gradients

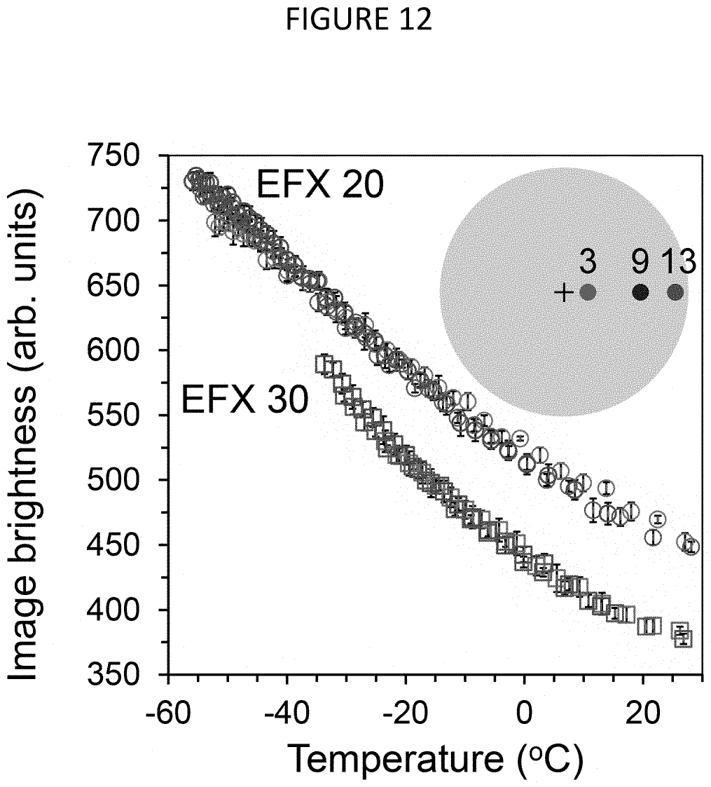

To determine the spatial resolution, a phantom composed of Ecoflex™ 00-30 silicone dielectric gel (Ecoflex 00-30) (Smooth-On, Inc., Macungie, Pa.) was surrounded with dry ice to make the edges colder than the center during the cool-down. The temperature at the edge and near the center was measured with MM compatible temperature sensors (e.g., miniature GaAs fiber optic bandgap spectral position sensor, TempSens by Opsens, Quebec, Canada). The magnetic resonance image brightness varied significantly from the center to the edge, and correlated with the temperature measurements (see FIG. 11) demonstrating that MRI can visually indicate temperature. Thus, both spatial and temperature resolution can be obtained directly from the image. A spatial resolution of about 5 mm with a temperature resolution of about 3° C. was observed in this example.

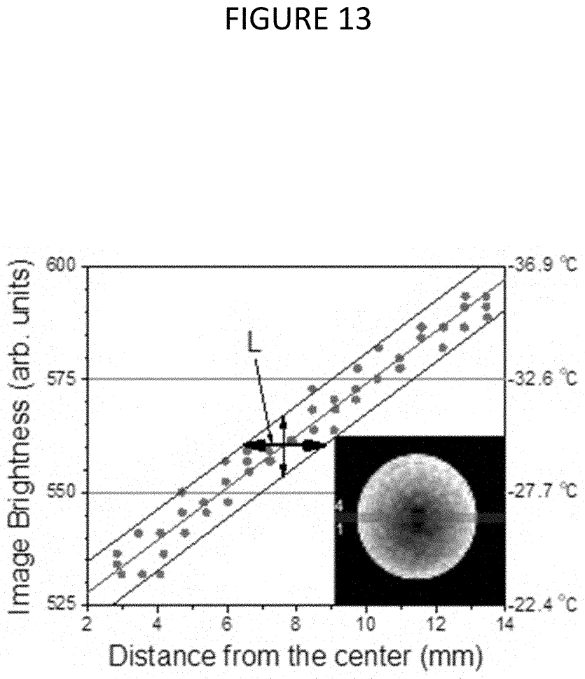

In another experiment, Ecoflex 00-30 and Ecoflex 00-20 silicone elastomer polymer phantoms were heated to a uniform temperature of 40° C., then dry ice was packed around each phantom, followed by measuring the MRI brightness (gradient echo sequence for T₁ weighting) as a function of position (radial distances of 3, 9 and 13 mm) every minute to produce a sequence of images. Next, temperature measurements were taken from the phantom following the same protocol that is used for the MM data acquisition. From this data, image brightness was obtained as function of temperature at each measured position. FIG. 12 shows a plot of brightness as a function of temperature for the two materials (Ecoflex 00-30 and Ecoflex 00-20) and three different positions, close to the center (3 mm), close to the middle (9 mm) and close to the edge (13 mm). Both materials exhibit similar behavior. For Ecoflex 00-20 the experiments were carried out down to −55° C., with useful results over the entire range. This shows that MRI brightness of these polymers can be used as a direct thermometer in MRI experiments, both above and below freezing temperatures.

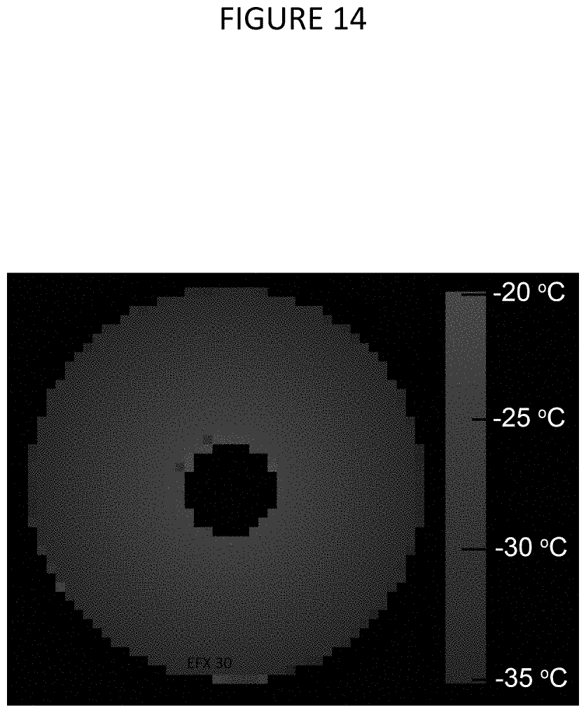

FIG. 13 shows the MM brightness measured at different locations at a particular acquisition time. The acquisition time determines the temperature distribution in the sample during the cooling as discussed above. The Y-axis scale on the right side of the FIG. 13 dot plot shows the temperature associated with a given brightness, which is shown on the left Y-axis. The data points are from a narrow slice of the left side of the smoothed MR image as indicated in the inset to FIG. 13. In addition, there is some error introduced by the finite measurement time due to changes in temperature over the measurement time (16 frames taken in 1 minute). The average temperature for each point is depicted in the dot plot.

The data presented in FIG. 13 enabled the determination of spatial resolution. Here, the effective spatial resolution, L=2.5 mm, is defined as the distance at which a temperature change of 3° C. or less can be clearly identified. Three degrees Celsius was selected as the temperature interval because that is what is often required in clinical applications.

### Example 5: Temperature Maps

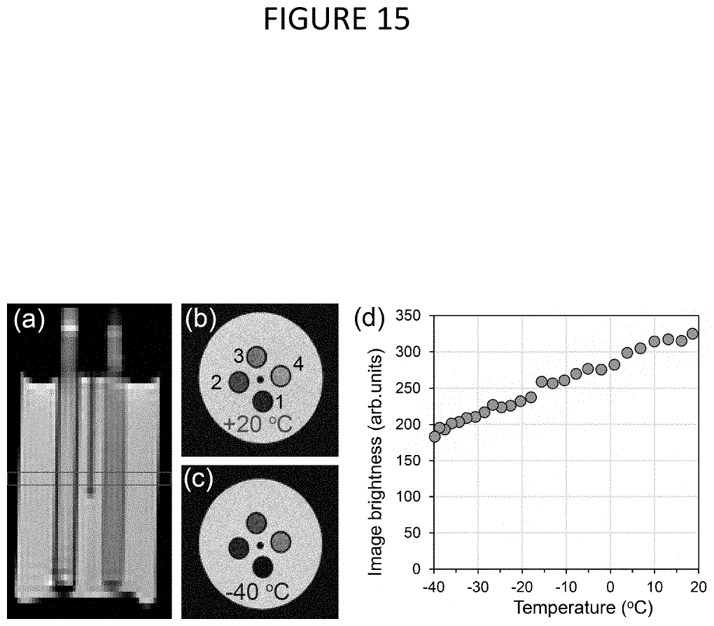

The original image in FIG. 11 show a number of artifacts (ringing) associated with a particular acquisition protocol resulting in the Gibbs phenomena. This is rectified by employing a numerical procedure such as smoothing. FIG. 14 shows a corrected false color image of the phantom depicted in FIG. 11 with a temperature color scale.

### Example 6: MRI Thermometry with Magnetic Ferrite Particle-Embedded Polymer

A magnetic ferrite particle-embedded polymer phantom (0.5 mM 3-micron Cu_{0.24}Zn_{0.76}Fe₂O₄ particle in Ecoflex silicone or Sylgard 527 dielectric gel) was warmed in a water bath to +40° C. and then transferred to a thermos filled with dry ice. The thermos with phantom was the placed into an MRI scanner's magnet bore to a previously defined position. Continuous imaging was then conducted according to the following MRI gradient echo sequence parameters: repetition time=0.236 s, echo time=3.4 ms, flip angle=20°, field of view=40×40 mm, spatial resolution=0.3 mm/pixel, slice thickness=4 mm, acquisition time=120 s. This sequence results in T₂*weighted images.

The experimental setup functioned in two modes: (1) with thick super-insulation made of aerogel surrounding the phantom to provide slow temperature drops (approximately 1° C./min) with a nearly uniform temperature across the phantom; and (2) without the super-insulation such that the phantom is in close thermal contact with the dry ice reservoir allowing for the occurrence of a strong temperature gradient across the phantom. MRI axial snapshots of the phantom were taken at these nonuniform gradient temperatures and the temperature inside the phantom was monitored locally in real time by a temperature controller with four sub-miniature GaAs sensors (TempSens by Opsens, Quebec, Canada).

FIG. 15a shows a representative MRI sagittal image of the phantom at about 20° C. FIGS. 15b and 15c show T₂* weighted axial images at +20° C. and −40° C., respectively. Here, all the MR images darken as the temperature is reduced. FIG. 15d presents the resulting brightness of the MR image as a function of temperature for the 0.5 mM Cu_{0.24}Zn_{0.76}Fe₂O₄ particle concentration. As the temperature increases, there is a gradual and near linear increase in brightness of the image. This temperature dependence on MR image brightness, in the form of an analytical function, can serve as a calibration formula to obtain absolute temperature.

### Example 7: Temperature Maps with Specific Temperature Intervals

FIG. 14 showed a false color image of the Ecoflex 00-30 phantom measured by MRI as originally shown in FIG. 11 after smoothing. FIG. 16 shows a different representation of FIG. 14, now with a temperature color scale and contours separated by three degrees. The asymmetry in temperatures at the top and bottom are more apparent in this representation than in FIG. 14. This example shows temperature intervals of three degrees, other temperature intervals, such as 2° C. or 4° C. are other useful visualizations.

### Example 8: MRI Signal Optimization

MRI intensity as a function of temperature was calculated using different flip angles (a.k.a. tip angles). The parameters for Dragon Skin material, which has a known dependence of T₁ as a function of temperature, as shown in FIG. 7 and Example 2, were used to simulate the changes in the MRI gradient echo signal strength with flip angles between 20° and 90° and a repetition time of TR=118 ms over a temperature range of 0° C. to −60° C. When the flip angle was set to 20°, a 28% change in the signal strength was observed with temperature. In contrast, when the flip angle was set to 70° under otherwise the same conditions, the percent change in signal strength was observed to be 166%.

Here, the percent change in intensity was calculated using Equation 2.

$$\frac{Intensity at - 60 ° C . - Intensity at 0 ° C .}{Intensity at 0 ° C .} \qquad Eq . 2$$

### Example 9: Temperature Maps from T1 Relaxation Time Values

Using standard MRI protocols and pulse sequences it is possible to obtain a direct map of T1 relaxation times values for regions within the human body. Experimental data show a near linear correlation of the T1 nuclear relaxation time with temperature in silicones. Hence, the measurement of T1 maps of silicone materials implanted into a tumor allows the direct creation of temperature maps for the tumor in cryoablations surgeries or other applications. This approach is machine independent and thus has an advantage that different MM systems can directly utilize this correlation for temperature determination without a calibration step. This method constitutes another method for determining temperature during cryoablation procedures using an implant or body of an appropriate material.

Further embodiments of the present invention can be described by the following methods:

Method 1. A method for determining the temperature of an object comprising the steps of:
a. placing temperature-stable hydrogen proton-rich material into an object;
b. cooling the object to ≤0° C.;
c. exposing the object to radio waves in a magnetic field; and
d. producing a magnetic resonance image of the temperature-stable hydrogen proton-rich material,
e. wherein the local level of brightness of the temperature-stable hydrogen proton-rich material indicates the spatial distribution of temperature in the object.

Method 2. The method 1 wherein the temperature-stable hydrogen proton-rich material is a biocompatible polymer and remains mechanically stable between room temperature and −65° C.

Method 3. The method 1 or 2 wherein the temperature-stable hydrogen proton-rich material has strong and monotonic temperature dependence of nuclear relaxation times when subjected to nuclear magnetic resonance scanning at temperatures between 0° C. and −65° C.

Method 4. Any one of methods 1-3 wherein the temperature-stable hydrogen proton-rich material comprises a silicone elastomer.

Method 5. The method 1 or 2 wherein the temperature-stable hydrogen proton-rich material has a narrow nuclear magnetic linewidth and weak temperature dependent nuclear relaxation times when subjected to nuclear magnetic resonance scanning at temperatures between 0° C. and −65° C.

Method 6. The method 5 wherein the temperature-stable hydrogen proton-rich material is doped with a plurality of magnetic particles that show a change in magnetization with a change in temperature over a temperature range of at least from about 37° C. to about −65° C. while under the magnetic field.

Method 7. The method 6 wherein the plurality of magnetic particles has an average diameter of about 5 nm to about 10 microns.

Method 8. The method 6 or 7 wherein each magnetic particle comprises an iron oxide doped with one or more metals selected from the group consisting of a 3d metal, a trivalent metal, and a divalent metal.

Method 9. Any one of methods 6-8 wherein each magnetic particle comprises an iron oxide doped with one or more metals selected from the group consisting of zinc, copper, manganese, magnesium, and yttrium.

Method 10. The method 6 or 7 wherein the magnetic particles comprise one or more of a ferromagnet, a ferrimagnet, a paramagnet, a canted antiferromagnet, a Permalloy doped with Cu, Gd, Gd doped with Cu, FeBO₃, rare-earth doped iron-oxide garnets, alloys of FeGd, and Co/Gd.

Method 11. Any one of methods 5-10 wherein the temperature-stable hydrogen proton-rich material is doped with about 0.05 mM to about 3 mM of said magnetic particles.

Method 12. Any one of methods 1-11 wherein the temperature-stable hydrogen proton-rich material is a filament having an aspect ratio ≥2.

Method 13. The method 12 wherein two or more filaments are placed into the object.

Method 14. The method 13 wherein one filament is placed at or near one side of the object and another filament is placed at or near the other side of the object.

Method 15. Any one of methods 1-14 wherein the temperature-stable hydrogen proton-rich material is coated onto a hypodermic needle.

Method 16. Any one of methods 1-14 wherein the temperature-stable hydrogen proton-rich material is formed as a hypodermic needle.

Method 17. Any one of methods 1-16 wherein the object is a tumor within a patient.

Method 18. Any one of methods 1-17 further comprising the step of adjusting a magnetic resonance imaging flip angle to increase temperature-dependent changes in the level of MR image brightness for given values of repetition and echo times.

Method 19. Any one of methods 1-17 further comprising the step of adjusting a magnetic resonance imaging repetition time to increase temperature-dependent changes in the level of MR image brightness for given values of flip angle and echo time.

Method 20. Any one of methods 1-17 further comprising the step of adjusting a magnetic resonance imaging echo time to increase temperature-dependent changes in the level of MR image brightness for given values of flip angle and repetition time.

Method 21. Any one of methods 1-17 further comprising the step of adjusting the magnetic resonance imaging repetition time and echo time to increase temperature-dependent changes in the level of MR image brightness for given value of flip angle.

Method 22. Any one of methods 1-17 further comprising the step of adjusting a magnetic resonance imaging flip angle and repetition time to increase temperature-dependent changes in the level of MR image brightness for given value of echo time.

Method 23. Any one of methods 1-17 further comprising the step of adjusting a magnetic resonance imaging flip angle and echo time to increase temperature-dependent changes in the level of MR image brightness for given value of repetition time.

Method 24. Any one of methods 1-17 further comprising the step of adjusting the magnetic resonance imaging repetition time, echo time, and flip angle to increase temperature-dependent changes in the level of MR image brightness.

Method 25. A method for killing hyperplastic cells comprising the steps of placing one or more filaments comprising a temperature-stable hydrogen proton-rich material into a tumor;
a. placing a probe into the tumor;
b. freezing the tumor by injecting gas at high pressure within the probe to a specific temperature ≤−10° C.;
c. determining the temperature of the tumor by imaging the one or more filaments by T₁, T₂, or T₂* nuclear magnetic resonance imaging; and
d. thawing the tumor,

wherein the brightness of the image of the one or more filaments correlates with the temperature the one of more filaments,

wherein the temperature-stable hydrogen proton-rich material is mechanically stable from room temperature to −65° C., and

wherein the temperature-stable hydrogen proton-rich material comprises a high abundance of hydrogen protons to enable the magnetic resonance image to be detectable and brighter than the surrounding target.

Method 26. The method 25 wherein the temperature-stable hydrogen proton-rich material comprises a polymer with monotonic temperature dependence of nuclear relaxation times T₁, T₂, or T₂* in the range of about 5 ms to about 1,500 ms over a range of temperatures to which the tumor is subjected.

Method 27. The method 25 or 26 wherein the polymer is a silicone elastomer.

Method 28. The method 25 or 26 wherein the polymer is a biocompatible polyepoxide.

Method 29. The method 25 wherein the temperature-stable hydrogen proton-rich material comprises: a polymer with a narrow NMR linewidth and weak temperature dependence in the range of about 0° C. to about −65° C.; and concentrations of magnetic particles of about 0.05 mM to about 3 mM of.

Method 30. The method of claim 29 wherein the magnetic particles comprise iron oxide doped with one or more metals selected from the group consisting of a 3d metal, a trivalent metal, and a divalent metal.

Method 31. The method 29 or 30 wherein the magnetic particles comprise iron oxide doped with one or more metals selected from the group consisting of zinc, copper, manganese, magnesium, and yttrium.

Method 32. The method 29 wherein the magnetic particles comprise one or more of a ferromagnet, a ferrimagnet, a paramagnet, a canted antiferromagnet, a Permalloy doped with Cu, Gd, Gd doped with Cu, FeBO3, rare-earth doped iron-oxide garnets, alloys of FeGd, and Co/Gd.

Further embodiments of the present invention can be described by the following systems:

System 1. A system for measuring the temperature of an object, the system comprising:
a. a magnetic resonance imaging (MRI) scanner; and
b. a hydrogen proton-rich filament.

System 2. The system 1, wherein the MRI scanner comprises a 0.2 Tesla to 7 Tesla magnet.

System 3. The system 1 or 2, wherein the MRI scanner comprises a 3 Tesla magnet.

System 4. Any one of systems 1-3 wherein the hydrogen proton-rich filament has an aspect ratio ≥2.

System 5. Any one of systems 1-4, wherein the hydrogen proton-rich filament exhibits monotonic temperature dependence of nuclear relaxation times T₁, T₂, or T₂* in the range of about 5 ms to about 1,500 ms over a temperature range of about 0° C. to −65° C. or over a temperature range of about +37° C. to +80° C.

System 6. Any one of systems 1-4, wherein the hydrogen proton-rich filament comprises a polymer having a narrow nuclear magnetic linewidth with weak temperature dependent nuclear relaxation times when subjected to nuclear magnetic resonance scanning at temperatures between 0° C. and −65° C. or at temperatures between +37° C. to +80° C.

System 7. The system 6, wherein the hydrogen proton-rich filament comprises magnetic particles that exhibit a change in magnetization with a change in temperature over a temperature range of at least from about +37° C. to about −65° C. or at least from about +37° C. to about +80° C. while under the magnetic field.

System 8. The system 6 or 7, wherein the magnetic particles have an average diameter of about 5 nm to about 10 microns.

System 9. any one of systems 6-8, wherein the magnetic particles comprise iron oxides.

System 10. The system 9 wherein the magnetic particles comprise iron oxides doped with one or more metals selected from the group consisting of a 3d metal, a trivalent metal, and a divalent metal.

System 11. The system 9 or 10 wherein each magnetic particle comprises an iron oxide doped with one or more metals selected from the group consisting of zinc, copper, manganese, magnesium, and yttrium.

System 12. Any one of systems 6-11, wherein the magnetic particles comprise Cu_{0.24}Zn_{0.76}Fe₂O₄.

System 13. Any one of systems 6-8 wherein the magnetic particles comprise one or more of a ferromagnet, a ferrimagnet, a paramagnet, a canted antiferromagnet, a Permalloy doped with Cu, Gd, Gd doped with Cu, FeBO3, rare-earth doped iron-oxide garnets, alloys of FeGd, and Co/Gd.

System 14. Any one of systems 32-13, wherein the hydrogen proton-rich filament comprises the magnetic particles at a concentration of about 0.05 mM to about 3 mM.

System 15. Any one of systems 11-14, wherein the hydrogen proton-rich filament comprises a biocompatible polymer.

System 16. Any one of systems 1-15, wherein the hydrogen proton-rich filament comprises a silicone elastomer or epoxy.

System 17. Any one of systems 1-16 further comprising a cryoablation probe and pressurized gas.

System 18. Any one of systems 1-17 further comprising a thermal ablation probe.

System 19. The system 13 further comprising a laser, wherein said thermal ablation probe is an optical fiber with a diffuser.

System 20. Any one of systems 1-19, wherein the object is a tumor.

System 21. Any one of systems 1-19, wherein the object is a cluster of atrioventricular cells.

System 22. Any one of systems 1-16, wherein the object is on or in a patient.

System 23. The system 17, wherein the cryoablation probe comprises a hydrogen proton-rich material that images as a bright material under nuclear magnetic resonance imaging at temperatures ≤0° C.

System 24. The system of claim 18, wherein the thermal ablation probe comprises a hydrogen proton-rich material that images as a bright material under nuclear magnetic resonance imaging at temperatures ≥+37° C.

Further embodiments of the present invention can be described by the following methods:

Method A1. A method for determining the temperature of an object comprising the steps of:
a. placing temperature-stable hydrogen proton-rich material into an object;
b. heating the object to ≥+37° C.;
c. exposing the object to radio waves in a magnetic field; and
d. producing a magnetic resonance image of the temperature-stable hydrogen proton-rich material,

wherein the level of brightness of the temperature-stable hydrogen proton-rich material indicates the temperature of the object.

Method A2. The method A1 wherein the temperature-stable hydrogen proton-rich material is a biocompatible polymer and remains mechanically stable at least between room temperature and +80° C.

Method A3. The method A1 or A2 wherein the temperature-stable hydrogen proton-rich material has strong and monotonic temperature dependence of nuclear relaxation times when subjected to nuclear magnetic resonance scanning at temperatures at least between +37° C. and +80° C.

Method A4. Any one of methods A1-A3 wherein the temperature-stable hydrogen proton-rich material comprises a silicone elastomer or a polyepoxide.

Method A5. The method A1 or A2 wherein the temperature-stable hydrogen proton-rich material has a narrow nuclear magnetic linewidth and weak temperature dependent nuclear relaxation times when subjected to nuclear magnetic resonance scanning at temperatures at least between +37° C. and +80° C.

Method A6. The method A5 wherein the temperature-stable hydrogen proton-rich material is doped with a plurality of magnetic particles that show a change in magnetization with a change in temperature over a temperature range of at least from about +37° C. and +80° C. while under the magnetic field.

Method A7. The method A6 wherein the plurality of magnetic particles has an average diameter of about 5 nm to about 10 microns.

Method A8. The method A5 or A6 wherein each magnetic particle comprises an iron oxide doped with one or more metals selected from the group consisting of a 3d metal, a trivalent metal, and a divalent metal.

Method A9. The methods A5-A8 wherein each magnetic particle comprises an iron oxide doped with one or more metals selected from the group consisting of zinc, copper, manganese, magnesium, and yttrium.

Method A10. Any one of methods A5-A9 wherein each magnetic particle comprises Cu_{0.35}Zn_{0.65}Fe₂O₄.

Method A11. The method A5 or A6 wherein the magnetic particles comprise one or more of a ferromagnet, a ferrimagnet, a paramagnet, a canted antiferromagnet, a Permalloy doped with Cu, Gd, Gd doped with Cu, FeBO3, rare-earth doped iron-oxide garnets, alloys of FeGd, and Co/Gd.

Method A12. Any one of methods A6-A11 wherein the temperature-stable hydrogen proton-rich material is doped with about 0.05 mM to about 3 mM of said magnetic particles.

Method A13. Any one of methods A1-A12 wherein the temperature-stable hydrogen proton-rich material is a filament having an aspect ratio ≥2.

Method A14. The method A13 wherein two or more filaments are placed into the object.

Method A15. The method A14 wherein one filament is placed at or near one side of the object and another other filament is placed at or near the other side of the object.

Method A16. Any one of methods A1-A14 wherein the temperature-stable hydrogen proton-rich material is coated onto a hypodermic needle or optical fiber.

Method A17. Any one of methods A1-A16 wherein the object is a tumor within a patient.

Method A18. Any one of methods A1-A17 further comprising the step of adjusting a magnetic resonance imaging flip angle to greater than 20 degrees to increase temperature-dependent changes in the level of brightness.

Method A19. Any one of methods A1-A17 further comprising the step of adjusting a magnetic resonance imaging flip angle to increase temperature-dependent changes in the level of MR image brightness for given values of repetition and echo times.

Method A10. Any one of methods A1-A17 further comprising the step of adjusting a magnetic resonance imaging repetition time to increase temperature-dependent changes in the level of MR image brightness for given values of flip angle and echo time.

Method A11. Any one of methods A1-A17 further comprising the step of adjusting a magnetic resonance imaging echo time to increase temperature-dependent changes in the level of MR image brightness for given values of flip angle and repetition time.

Method A12. Any one of methods A1-A17 further comprising the step of adjusting the magnetic resonance imaging repetition time and echo time to increase temperature-dependent changes in the level of MR image brightness for given value of flip angle.

Method A13. Any one of methods A1-A17 further comprising the step of adjusting a magnetic resonance imaging flip angle and repetition time to increase temperature-dependent changes in the level of MR image brightness for given value of echo time.

Method A14. Any one of methods A1-A17 further comprising the step of adjusting a magnetic resonance imaging flip angle and echo time to increase temperature-dependent changes in the level of MR image brightness for given value of repetition time.

Method A15. Any one of methods A1-A17 further comprising the step of adjusting the magnetic resonance imaging repetition time, echo time, and flip angle to increase temperature-dependent changes in the level of MR image brightness.

Method B1. A method for killing hyperplastic cells comprising the steps of:
a. placing one or more filaments comprising a temperature-stable hydrogen proton-rich material into a tumor;
b. heating the tumor to a specific temperature ≥+65° C.; and
c. determining the local temperature within the tumor by imaging the one or more filaments using MRI with T₁, T₂, or T₂* weightings

wherein the brightness of the image of the one or more filaments correlates with the temperature the one of more filaments,

wherein the temperature-stable hydrogen proton-rich material is mechanically stable from +37° C. to +80° C., and

wherein the temperature-stable hydrogen proton-rich material comprises a high abundance of hydrogen protons to enable the magnetic resonance image to be detectable and brighter than the surrounding target.

Method B2. The method B1 wherein the temperature-stable hydrogen proton-rich material comprises a polymer with monotonic temperature dependence of nuclear relaxation times T₁, T₂, or T₂* in the range of about 5 ms to about 1,500 ms over a range of temperatures to which the tumor is subjected.

Method B3. The method B1 or B2 wherein the polymer is a silicone elastomer.

Method B4. The method B1 or B2 wherein the polymer is a biocompatible polyepoxide.

Method B5. The method B1 wherein the temperature-stable hydrogen proton-rich material comprises: a polymer with a narrow NMR linewidth and weak temperature dependence in the range of about +37° C. to about +80° C.; and magnetic particle concentrations of about 0.05 mM to about 3 mM.

Method B6. The method B5 wherein said magnetic particles comprise iron oxides.

Method B7. The method B5 or B6 wherein said magnetic particles comprise iron oxides doped with one or more metals selected from the group consisting of a 3d metal, a trivalent metal, and a divalent metal.

Method B8. Any one of methods B5-B7 wherein each magnetic particle comprises an iron oxide doped with one or more metals selected from the group consisting of zinc, copper, manganese, magnesium, and yttrium.

Method B9. The method B5 wherein the magnetic particles comprise one or more of a ferromagnet, a ferrimagnet, a paramagnet, a canted antiferromagnet, a Permalloy doped with Cu, Gd, Gd doped with Cu, FeBO3, rare-earth doped iron-oxide garnets, alloys of FeGd, and Co/Gd.

Method C1. A method for monitoring the killing hyperplastic cells comprising the steps of:
a. placing one or more filaments comprising a temperature-stable hydrogen proton-rich material into a tumor;
b. placing a probe into the tumor;
c. applying a killing temperature to the tumor; and
d. determining the local temperature within the tumor by imaging the one or more filaments using MRI with T₁, T₂, or T₂* weightings;

wherein the brightness of the image of the one or more filaments correlates with the temperature the one of more filaments,

wherein the temperature-stable hydrogen proton-rich material is mechanically stable from room temperature to −65° C., or from +37° C. to +80° C., and

wherein the temperature-stable hydrogen proton-rich material comprises a high abundance of hydrogen protons to enable the magnetic resonance image to be detectable and brighter than the surrounding target.

Method C2. The method C1 wherein the killing temperature is ≤−10° C.

Method C3. The method C1 wherein the killing temperature is ≥+40° C.

“Substantially” or “about” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

1. A method for killing cells in a tumor comprising the steps of:
a) placing one or more filaments comprising a temperature-stable hydrogen proton-rich material into the tumor;
b) placing a probe into the tumor;
c) freezing the tumor to a specific temperature ≤−10° C. by injecting gas at high pressure within the probe;
d) determining the temperature of the tumor by imaging the one or more filaments by T₁, T₂, or T₂* nuclear magnetic resonance imaging; and
e) thawing the tumor,
wherein the brightness of the image of the one or more filaments correlates with the temperature of the one of more filaments,
wherein the temperature-stable hydrogen proton-rich material is mechanically stable from room temperature to −65° C., and
wherein the temperature-stable hydrogen proton-rich material comprises a high abundance of hydrogen protons to enable the magnetic resonance image to be detectable and brighter than the surrounding target.

2. The method of claim 1 wherein the temperature-stable hydrogen proton-rich material comprises a polymer with monotonic temperature dependence of nuclear relaxation times T₁, T₂, or T₂* in the range of about 5 ms to about 1,500 ms over a range of temperatures to which the tumor is subjected.

3. The method of claim 2 wherein the polymer is a silicone elastomer.

4. The method of claim 2 wherein the polymer is a biocompatible polyepoxide.

5. The method of claim 1 wherein the temperature-stable hydrogen proton-rich material comprises: a polymer with a narrow NMR linewidth and weak temperature dependence in the range of about 0° C. to about −65° C.; and magnetic particles in concentrations of about 0.05 mM to about 3 mM.

6. The method of claim 5 wherein the magnetic particles comprise iron oxide doped with one or more metals selected from the group consisting of a 3d metal, a trivalent metal, and a divalent metal.

7. The method of claim 5 wherein the magnetic particles comprise iron oxide doped with one or more metals selected from the group consisting of zinc, copper, manganese, magnesium, and yttrium.

8. The method of claim 5 wherein the magnetic particles comprise one or more of a ferromagnet, a ferrimagnet, a paramagnet, a canted antiferromagnet, a Permalloy doped with Cu, Gd, Gd doped with Cu, FeBO3, rare-earth doped iron-oxide garnets, alloys of FeGd, and Co/Gd.

9. A method for killing cells in a tumor comprising the steps of:
a) placing one or more filaments comprising a temperature-stable hydrogen proton-rich material into a tumor;
b) heating the tumor to a specific temperature ≥+65° C.; and
c) determining the temperature of the tumor by imaging the one or more filaments by T₁, T₂, or T₂* nuclear magnetic resonance imaging,
wherein the brightness of the image of the one or more filaments correlates with the temperature the one of more filaments,
wherein the temperature-stable hydrogen proton-rich material is mechanically stable from +37° C. to +80° C., and
wherein the temperature-stable hydrogen proton-rich material comprises a high abundance of hydrogen protons to enable the magnetic resonance image to be detectable and brighter than the surrounding target.

10. The method of claim 9 wherein the temperature-stable hydrogen proton-rich material comprises a polymer with monotonic temperature dependence of nuclear relaxation times T₁, T₂, or T₂* in the range of about 5 ms to about 1,500 ms over a range of temperatures to which the tumor is subjected.

11. The method of claim 10 wherein the polymer is a silicone elastomer.

12. The method of claim 10 wherein the polymer is a biocompatible polyepoxide.

13. The method of claim 9 wherein the temperature-stable hydrogen proton-rich material comprises: a polymer with a narrow NMR linewidth and weak temperature dependence in the range of about +37° C. to about +80° C.; and magnetic particles in concentrations of about 0.05 mM to about 3 mM.

14. The method of claim 13 wherein said magnetic particles comprise iron oxides.

15. The method of claim 13 wherein said magnetic particles comprise iron oxides doped with one or more metals selected from the group consisting of a 3d metal, a trivalent metal, and a divalent metal.

16. The method of claim 13 wherein each magnetic particle comprises an iron oxide doped with one or more metals selected from the group consisting of zinc, copper, manganese, magnesium, and yttrium.

17. The method of claim 13 wherein the magnetic particles comprise one or more of a ferromagnet, a ferrimagnet, a paramagnet, a canted antiferromagnet, a Permalloy doped with Cu, Gd, Gd doped with Cu, FeBO3, rare-earth doped iron-oxide garnets, alloys of FeGd, and Co/Gd.

18. A method for killing cells in a tumor, the method comprising the steps of:
a) placing one or more filaments comprising a temperature-stable hydrogen proton-rich material into the tumor;
b) placing a probe into the tumor;
c) applying a killing temperature to the tumor; and
d) determining the temperature of the tumor by imaging the one or more filaments by T₁, T₂, or T₂* nuclear magnetic resonance imaging,
wherein the brightness of the image of the one or more filaments correlates with the temperature the one of more filaments,
wherein the temperature-stable hydrogen proton-rich material is mechanically stable from room temperature to −65° C., or from +37° C. to +80° C., and
wherein the temperature-stable hydrogen proton-rich material comprises a high abundance of hydrogen protons to enable the magnetic resonance image to be detectable and brighter than the surrounding target.

19. The method of claim 18 wherein the killing temperature is ≤−10° C.

20. The method of claim 18 wherein the killing temperature is ≥+40° C.

21. A method for killing cells in a tumor, the method comprising the steps of:
a. Placing a filament comprising a temperature-stable hydrogen proton-rich material into the tumor;
b. placing a probe into the tumor;
c. altering the temperature of the tumor by heating or cooling the probe;
d. determining the temperature of a portion of the tumor by measuring the T1 relaxation time of the filament; and
e. altering the temperature of the probe to alter the temperature of the tumor to a killing temperature.

22. The method of claim 21 wherein the temperature-stable hydrogen proton-rich material comprises a polymer with monotonic temperature dependence of nuclear relaxation time T₁ in a range of +37° C. to about +80° C.

23. The method of claim 21 wherein the temperature-stable hydrogen proton-rich material comprises a polymer with monotonic temperature dependence of nuclear relaxation time T₁ in a range of 0° C. to about −65° C.

24. The method of claim 21 wherein the temperature-stable hydrogen proton-rich material is a silicone elastomer.

25. The method of claim 1 further comprising the step of adjusting a magnetic resonance imaging flip angle to increase temperature-dependent changes in the level of brightness of the image of the one or more filaments.

26. The method of claim 9 further comprising the step of adjusting a magnetic resonance imaging flip angle to increase temperature-dependent changes in the level of brightness of the image of the one or more filaments.

27. The method of claim 18 further comprising the step of adjusting a magnetic resonance imaging flip angle to increase temperature-dependent changes in the level of brightness of the image of the one or more filaments.
